SEQUENCE LISTING (8 sequences)

SEQ ID NO: 1 - DNA, 38 bp, Artificial Sequence
  Description of Artificial Sequence Synthetic oligonucleotide
aagcttgatcgaactgaccgcccgcggcccgtggatcc

SEQ ID NO: 2 - DNA, 38 bp, Artificial Sequence
  Description of Artificial Sequence Synthetic oligonucleotide
ggatccacgggccgcgggcggtcagttcgatcaagctt

SEQ ID NO: 3 - RNA, 38 nt, Artificial Sequence
  Description of Artificial Sequence Synthetic oligonucleotide
aagcuugaucgaacugaccgcccgcggcccguggaucc

SEQ ID NO: 4 - DNA, 38 bp, Artificial Sequence
  Description of Combined DNA/RNA Molecule Synthetic oligonucleotide
ggatccacgggccgcgggcggucagttcgatcaagctt

SEQ ID NO: 5 - RNA, 38 nt, Artificial Sequence
  Description of Artificial Sequence Synthetic oligonucleotide
ggauccacgggccgcgggcggucaguucgaucaagcuu

SEQ ID NO: 6 - RNA, 13 nt, Artificial Sequence
  Description of Artificial Sequence Synthetic oligonucleotide
ggaugucgaucac

SEQ ID NO: 7 - RNA, 10 nt, Artificial Sequence
  Description of Artificial Sequence Synthetic oligonucleotide
ggagcgacac

SEQ ID NO: 8 - RNA, 10 nt, Artificial Sequence
  Description of Artificial Sequence Synthetic oligonucleotide
ggacgacacc

(19) United States
(12) Patent Application Publication
(10) Pub. No.: US 2004/0086918 A1
(43) Pub. Date: May 6, 2004

(54) Macromolecular protection assay

(76) Inventors: Zvi Loewy, Fair Lawn, NJ (US); Wayne Chaung, New Hyde Park, NY (US); Raveendran Pottathil, La Jolla, CA (US)

Correspondence Address: FROMMER LAWRENCE & HAUG, 745 FIFTH AVENUE- 10TH FL., NEW YORK, NY, 10151, US

(21) Appl. No.: 10/627,561

(22) Filed: Jul. 25, 2003

Related U.S. Application Data
(60) Provisional application No. 60/398,685, filed on Jul. 26, 2002.

(51) Int. Cl. C12Q 1/68; G01N 33/53

(52) U.S. Cl. 435/6; 435/7.1

(57) ABSTRACT

The present invention relates to a novel method for the detection and characterization of an unknown nucleic acid-binding protein from a biological sample. More particularly, the method of the instant invention includes the steps of forming nucleic acid-protein complexes, the subsequent selective degradation of of unbound nucleic acid molecules, the detection of nucleic acid-protein complexes, and finally, the characterization of the nucleic acid-binding protein. Additionally, compositions and methods are provided in the present invention for detecting and characterizing unknown nucleic acid-binding proteins from biological samples using immobilized nucleic acid molecules in reaction vessels having a multitude of wells.

### RELATED APPLICATIONS/PATENTS & INCORPORATION BY REFERENCE

This application claims the benefit of U.S. Provisional Application Serial No. 60/398,685, filed on Jul. 26, 2002. Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### FIELD OF THE INVENTION

The present invention relates to a novel method for the detection and characterization of unknown nucleic acid-binding proteins from biological samples.

### BACKGROUND

DNA-binding proteins serve fundamentally important functions for all living organisms and play major roles in gene regulation, splicing, modulation, recombination, repair, replication and transcription. Given their fundamental roles in the cell, particularly in the regulation of gene expression, DNA-binding proteins, for example, transcription factors, are inextricably linked to human disease and cancer. Further, genetic variations in DNA-binding protein-encoding genes in human populations form at least one basis for genetic-linked variations in human responses to drugs and other pharmaceutical agents. Thus, the study of DNA-binding proteins is of fundamental interest to both academic scientists and pharmaceutical and biotechnology researchers.

RNA-binding proteins also serve important roles in cells and are of growing interest to both the academic and industrial sectors. For example, ribonucleoprotein particles (RNP) are protein-RNA complexes which direct a variety of processes relating to the protection, stabilization, packaging, and transport of RNA. Another example of RNA-binding proteins involves proteins that bind to catalytic RNAs. These RNA molecules fold into specific three-dimensional structures and can become complexed with specific proteins, which catalyze a variety of specific reactions. For example, catalytic RNAs are involved in carrying out RNA splicing reactions in nucleii of eukaryotic cells. An unrelated, but ubiquitous example of RNA-binding proteins are the protein subunits of the ribosome, which are found in all organisms. This structure, which directs the protein translation process, contains a number of RNA-binding proteins complexed with at least three different RNA molecules that are involved in the translation process. In addition to their fundamental roles in prokaryotic and eukaryotic RNA protection, processing, transport, and expression, RNA-binding proteins are also important components of some viruses.

The study of DNA- and RNA-binding proteins is highly prevalent in life science research and encompasses many fields of research including molecular biology, molecular genetics, biophysical protein chemistry, genomics, and proteomics. Further, given their link to human disease, these proteins are of immense interest to the pharmaceutical industry. For example, the human tumor suppressor gene, p53, encodes a eukaryotic DNA-binding protein that is known to be involved in cancer formation. Roughly, about 55 percent of all human cancers from many different cell or tissue types suffer mutations in both alleles of the p53 gene. Those who have inherited such mutations are highly prone to developing cancer over their lifetimes. Specifically, the p53 protein is a transcription factor, which regulates the expression of a large number of genes having a variety of functions. High levels of a mutant p53 protein in a cell leads to the overexpression of these genes, which in turn leads to unregulated cellular growth and thus, the potential for cancer. Targeting the p53 protein with appropriately designed small-molecule drugs represents a potential strategy for the pharmaceutical treatment of diseases such as cancer. Likewise, other DNA- and RNA-binding proteins can be similarly targeted to treat human diseases that involve improperly regulated genes.

To achieve a greater understanding of the processes under control of DNA- and RNA-binding proteins, particularly disease manifestation, progress may be influenced by at least the following two basic research directives. Firstly, the continued study of known or previously identified DNA- and RNA-binding proteins should be pursued to further understand the regulation and interactions of the many complex genetic networks and pathways that constitute cells, tissues, and organisms, including humans. Secondly, unknown or novel DNA- and RNA-binding proteins need to be identified and placed into the genetic framework already set in place. For example, a set of genes that are linked to cancer might be the subject of study. Of fundamental interest would be to understand the regulation of disease-related genes and the genetic basis for the manifestation of the associated disease. Addressing these questions may necessitate a better understanding of the transcriptional control of the genes at issue, i.e. which transcription factors are involved, which genes are regulated, and how the regulation is achieved. A better understanding of the genetic regulation of disease-related genes, as well as the transcription factors that control their expression, may facilitate the downstream identification and design of novel therapeutics.

Traditional approaches to studying DNA- and RNA-binding proteins, including transcription factors, has involved the use of various analytical tools such as gel-shift mobility assays, blotting, and footprinting. Although these techniques represent well established methods in the art for studying DNA- and RNA-binding proteins, their applications relate to the study of a specific molecular interaction between a target nucleic acid-binding sequence and a DNA- or RNA-binding protein. That is, they are used to study how known DNA- and RNA-binding proteins interact with a cognate nucleic acid-binding sequence. However, these methods have limitations that decrease their usefulness in detecting unknown or novel DNA- and RNA-binding proteins. In general, the methods currently used in the art lack adequate efficiency, speed, ease of use, and/or affordability for use in detecting unknown binding proteins, particularly in a high-throughput and scalable manner.

Further, the current methods known in the art are not amendable to automation, which limits their usefulness in genomics- and proteomics-based applications. Since genomics- and proteomics-based science is at the forefront of drug discovery processes in the pharmaceutical industry, improved methods for studying DNA- and RNA-binding proteins would be highly desirable. Examples of methods for studying nucleic acid-binding proteins can be found in the following documents: Technical Bulletin No. 137, “Core Footprinting System”, Promega Corporation (1999); R. Rapley editor, “The Southwestern Assay”, The Nucleic Acid Protocols Handbook p.773-782 (2000); R. Rapley editor, “The Gel Shift Assay for the Analysis of DNA-Protein Interactions”, The Nucleic Acid Protocols Handbook p.745-756 (2000); R. Rapley editor, “DNase I Footprinting”, The Nucleic Acid Protocols Handbook p.729-735 (2000); and Moss, “DNA-Protein Interactions,” Humana Press, volume 148 in Methods in Molecular Biology (2001).

The gel-shift mobility assay is a method for the analysis of DNA-protein interactions, which has also been adapted for the analysis of RNA-binding proteins. In this assay, radiolabeled nucleic acid and protein are mixed together, the solution is subjected to electrophoresis through a polyacrylamide gel, and the gel is analyzed by autoradiography of the radiolabeled nucleic acid. Binding of the protein to the nucleic acid can result in a complex that has a different electrophoretic mobility from the free nucleic acid. Separation of the complex from the free nucleic acid is indicative of an interaction between the nucleic acid and the protein. Although this is a relatively simple approach to studying nucleic acid-binding reactions, which could be used to detect nucleic acid proteins, it relies on gel electrophoresis. The dependence upon gel electrophoresis is a limitation of the method causing it to be slow, time-consuming, and labor-intensive. Further, it is not amendable to high-throughput, automated methods for detection of nucleic acid-binding proteins.

Gel electrophoresis imposes limitations on the number of samples that can be analyzed at any given time and thus, prevents the methods from being easily scaled-up or carried out in a high-throughput manner. Further, the current methods are relatively unreliable. For example, in the case of the gel-shift assay, not all proteins will form a nucleic acid-protein complex that will be distinguishable as a separate band on a gel. Further, most, if not all of the steps of the gel-shift assay and other current methods in the art are performed manually, which can lead to undesirable error and inconsistency between experiments. Automation is not realistically feasible for the gel-shift assay or any of the other methods available in the art.

Blotting techniques have also been developed to study nucleic acid-binding proteins. One such technique is Southwestern blotting. Using this method, proteins of crude cell extracts are first separated by SDS polyacrylamide gel electrophoresis (including 2-dimensional electrophoresis) and blotted onto a nitrocellulose membrane. Then, specific nucleic acid-binding proteins of interest are detected by incubating the membrane with a labeled nucleic acid probe that is derived from the protein binding site. It has also been developed as a preliminary step to set up conditions for in situ detection of DNA-binding proteins expressed by recombinant bacteriophages or to select DNA sequences recognized by a specific DNA-binding protein. Although this method can be utilized to detect nucleic acid-binding proteins from protein samples, it suffers from similar limitations to the technique of gel-shift mobility assay, namely, the requirement of gel electrophoresis, use of radioactivity, lack of scalability, lack of high-throughput potential, and lack of potential for automation.

Footprinting is a technique used to study DNA-protein and RNA-protein interactions. The technique was originally developed to study the sequence-specific binding of proteins to DNA. In this technique, a suitable uniquely end-labeled DNA fragment is allowed to interact with a given DNA-binding protein. The resulting protein-DNA complex is then partially digested with DNase I. The bound protein protects the region of the DNA with which it interacts from attack by DNase I. Subsequent molecular weight analysis of the degraded DNA by electrophoresis and autoradiography identifies the region of protection as a gap in the otherwise continuous background of digestion products. The technique can be used to determine the site of interaction of most sequence-specific DNA-binding proteins, but has been most extensively applied to the study of transcription factors.

In addition to DNase I-based footprinting, the footprinting technique has been adapted for use with alternative DNA-degrading enzymes, such as DNA N-glycosylases. For example, Devchand et. al. (Ann NY Acad Sci, 1994, 726:309-11), used uracil DNA N-glycosylase in footprinting studies to analyze the binding interaction between the tet repressor and its binding site, the tet operator sequence. In addition, Speck et. al. (Nucleic Acids Research, 1997, 25:3242-3247) described the use of uracil DNA N-glycosylase in footprinting studies to explore the specific interaction between the DnaA protein, a replication protein, and its chromosomal binding site, the DnaA box. These studies however, are focused on the analysis of the molecular interaction between a specific DNA sequence and a known protein. These studies do not relate to the use of DNA N-glycosylase-based footprinting techniques as an assay to characterize unknown DNA-binding proteins of interest from protein samples, but rather to study the specific interactions between a known DNA sequence and a known protein.

Furthermore, DNA footprinting techniques, regardless of the enzyme used in the assay, are strictly reliant on gel electrophoresis, making the technique labor-intensive, slow, inefficient, and difficult to automate or use in a high-throughput manner. Information relevant to the use of uracil N-glycosylase as a tool of molecular biology can also be found in U.S. Pat. Nos. 6,004,745, 5,888,795, 5,035,996, 6,048,696, 6,190,865, 5,770,370, 6,165,726, 6,090,553, and 5,962,225. However, these methods are either unrelated to the study of DNA-and RNA-binding proteins or fail to overcome the limitations of DNA footprinting, for example, that gel electrophoresis remains a requirement.

The methods above have been developed for studying the specific molecular interactions between nucleic acids and predetermined proteins. As such, the current methods are not suitable for high-throughput screening of unknown nucleic acid-binding proteins. Further, current technologies are limited by the number of protein samples which can feasibly be screened at any given time since their methods are cumbersome and labor-intensive. For example, DNA footprinting, which is a well-known technique in the art for studying DNA-binding proteins, requires a number of steps that are labor-intensive, such as, radiolabeling of DNA molecule comprising DNA-binding site, precipitation of DNA fragments generated from enzyme digestion, and resolution of DNA fragments using gel electrophoresis. Due to the requirement of these steps, DNA footprinting is not easily amendable to processing a large number of samples in parallel and thus, is not easily adapted to be high-throughput.

Given the importance of studying DNA- and RNA-binding proteins, their relevance to human disease, their potential as targets of therapeutic intervention, and the limitations of current methods in the art, there is a need for a method of studying nucleic acid-binding proteins that allows for the detection of novel nucleic acid-binding proteins that is easier to use, faster, more efficient, more effective, safer, and scalable. Further, there is a need for a method of detecting nucleic acid-binding proteins that is more reliable and has a higher degree of sensitivity than the methods currently known in the art. Further still, there is a need for such a method to be employed in a high-throughput format that can be fully or partially automated.

### SUMMARY

The present invention provides a reliable, efficient, rapid, and scalable method to detect an unknown DNA- or RNA-binding protein in a biological sample. The method is more reliable than prior methods and has a relatively high degree of sensitivity. The method is adaptable to processing a plurality of reactions in parallel, can be employed in a high throughput format, and is compatible with techniques of automation known in the art. Further still, the method can employ but does not require gel electrophoresis techniques, or any type of fractionation technique, to detect nucleic acid-binding proteins.

One or more objects of the instant invention can be to provide a novel method for the detection of unknown nucleic acid-binding proteins from biological samples; to provide a novel method for the detection and characterization of unknown nucleic acid-binding proteins from biological samples; to provide a novel method for the detection of unknown nucleic acid-binding proteins from biological samples not requiring the use of gel electrophoresis; to provide a novel method for the detection of unknown nucleic acid-binding proteins using a process that enriches for nucleic acid-protein binding complexes; to provide a novel method for the detection of unknown nucleic acid-binding proteins from biological samples in a high-throughput manner wherein a plurality of nucleic acid-binding proteins are detected using reaction vessels comprising a plurality of wells, such as, microtiter plates; to provide a novel method for the detection of unknown nucleic acid-binding proteins from biological samples that is partially or fully automated; to provide a kit comprising components necessary to carry out the method of the invention; to provide a kit comprising components necessary to carry out the method of the instant invention and an array of protein samples derived from different types of cells and/or tissues in order to elucidate cellular, genetic, and regulatory pathways; to provide a novel method for the detection of an unknown DNA-binding protein from a biological sample using a procedure to selectively degrade unbound DNA using a DNA N-glycosylase; to provide a novel method for the detection of an inhibitor of a nucleic acid-binding protein from a chemical sample; and to provide a method for validating the detection of a nucleic acid-binding protein by successively repeating the method of the instant invention under conditions of progressively increasing stringency carried out in a high-throughput manner.

In a preferred embodiment of the present invention, an unknown nucleic acid-binding protein is detected from a protein sample that potentially contains a nucleic acid-binding protein by first contacting a nucleic acid molecule comprising a nucleic acid-binding sequence with the protein sample under conditions sufficient to form a binding complex, such that the binding complex protects bound nucleic acid from degrading. The nucleic acid can be DNA or RNA. Next, the binding complex is subjected to nucleic acid degradation conditions which degrade any unbound nucleic acid molecules. Lastly, the bound nucleic acid that has not degraded is detected, wherein the detection of intact, bound nucleic acid indicates a nucleic acid-binding protein. The detected nucleic acid-binding protein can then be characterized using techniques known to the skilled artisan including immunodetection, mass spectrometry, amino acid sequencing, and enzymatic digestion of the DNA-binding protein. Detection of the nucleic acid-binding protein does not require gel electrophoresis or any comparable type of fractionation procedure known to the skilled artisan. Advantageously, detection of the nucleic acid-binding protein by this method does not require prior knowledge of the protein (e.g. knowledge of the DNA-binding domain or nucleic acid-binding sequence).

In one embodiment of the present invention, the method described above is carried out in a high-throughput manner, wherein a plurality of protein samples are processed in parallel. In a further embodiment, the method above is fully or partially automated, such that all or some of the steps of liquid handling, protein sample delivery, nucleic acid degradation, detection of bound nucleic acid, and characterization of the unknown nucleic acid-binding protein are carried out in a hands-free manner. In one aspect of the invention the method of the instant invention is performed utilizing microwell plates, e.g. microtiter plates.

In yet another preferred embodiment, a nucleic acid molecule, comprising a nucleic acid-binding sequence, has a chemical modification, such as incorporation of one or more uracil in place of one or more thymine, which enables it to be degraded by an enzyme. In one embodiment, the nucleic acid molecule is treated with a DNA N-glycosylase, such as uracil DNA N-glycosylase. The DNA N-glycosylase catalyzes the removal of the nucleotide bases that have the chemical modification from unbound nucleic acid molecules, which creates an apurinic/apyrimidinic (AP) site. Next, a break in the nucleic acid molecule at each AP site is introduced by exposing the DNA molecule to heat and/or alkali conditions. In another preferred embodiment, the nucleic acid molecule is treated with an AP lyase, which catalyzes the cleavage of a phosphodiester bond on the 3′ or 5′ side of an AP site. In this embodiment, the AP lyase preferably has a DNA N-glycosylase activity such that the AP site is introduced at chemically modified nucleotide bases. Likewise, a break in the nucleic acid molecule at each AP site is introduced by exposing the DNA molecule to heat and/or alkali conditions.

In another preferred embodiment, the method of the present invention comprises obtaining a protein sample potentially containing an unknown DNA-binding protein and contacting a DNA molecule, comprising a DNA-binding sequence, a label, and a chemical modification to one or more nucleotides comprising cytosine, thymine, adenine or guanine, with the protein sample under conditions sufficient to form a binding complex. The binding complex comprises the DNA-binding protein and a bound DNA molecule, wherein the formation of the binding complex protects the DNA molecule from degradation. Next, the binding complex is subjected to DNA degradation conditions, which degrades any unbound DNA molecules. Finally, the bound DNA is detected, wherein detecting said bound DNA indicates a DNA-binding protein. The detected DNA-binding protein can then be characterized using techniques known to the skilled artisan including immunodetection, mass spectrometry, amino acid sequencing, and enzymatic digestion of the nucleic acid-binding protein. The detection of the DNA-binding protein does not require gel electrophoresis or any similar type of fractionation procedure known to the skill artisan.

In yet another preferred embodiment, the method of the instant invention comprises obtaining one or more protein samples, wherein each protein sample potentially contains at least one unknown DNA-binding protein and combining said protein sample with at least one DNA molecule, comprising a DNA-binding sequence and a label, in one or more wells of a reaction vessel comprising a plurality of wells, such as, a microtiter plate. In one preferred embodiment, the reaction vessel is a strip well unit, comprising a linear arrangement of reaction wells and made from any known materials used in the art, such as, plastic. In another preferred embodiment, the reaction vessel is a planar well unit, comprising a flat surface having an array of reaction wells and made from any known materials used in the art, such as, plastic. In a still further preferred embodiment of the invention, the reaction vessel is a chip well unit, comprising an array of small-volume wells. The chip well unit can be made from any known materials used in the art, for example, plastic or glass, and can be obtained commercially, such as from Affymetrix, Inc. (Santa Clara, Calif.).

Next, the DNA molecule and the protein sample in the reaction vessel are exposed to conditions sufficient to form a binding complex, comprising the DNA-binding protein and a bound DNA molecule, wherein the binding complex protects the bound DNA molecule or fragment thereof from degradation. Then, the binding complex is subjected to DNA degradation conditions, wherein any unbound DNA molecules are degraded. Lastly, any bound DNA is detected, wherein detecting said bound DNA indicates the presence of a DNA-binding protein. The present embodiment can be carried out in a high-throughput manner.

In one embodiment of the instant invention, the DNA or nucleic acid molecule comprising the binding site for a nucleic acid-binding protein is bound or affixed to the surface of the reaction vessel, such as, for example, a well of a microtiter plate, during the time over which the method of the present invention is carried out, namely, the detection of unknown nucleic acid-binding proteins from biological samples. The present invention contemplates any method known to one of ordinary skill in the art to carry out the binding or affixing of the DNA or nucleic acid molecule of the invention. For example, the surface of a reaction vessel, such as a well of a microwell plate, can be coated with the protein streptavidin whereas the DNA or nucleic acid can be prepared having at least one attached molecule of biotin. The biotin is a ligand of streptavidin. Thus, the biotin-labeled DNA or nucleic acid molecule will bind to the surface of the well of the microwell plate vis-a-vis the streptavidin coated on the surface of the well. One of skill in the art will appreciate that the binding or affixing of the DNA or nucleic acid molecule to the reaction vessel surface, for example, to a well of a microtiter plate, is advantageous to the extent that intact, non-degraded DNA or nucleic acid can be detected more easily since it remains attached to the reaction vessel surface.

In a further embodiment, the DNA is bound to magnetic microparticles in a well of a reaction vessel comprising a plurality of wells, such as a microtiter plate, to facilitate liquid handling and automation.

In yet another preferred embodiment of the present invention, an inhibitor of a DNA-binding protein can be identified. In this embodiment, one or more protein samples potentially containing one or more unknown DNA-binding proteins is obtained. In addition, one or more chemical samples potentially containing one or more inhibitors of the DNA-binding protein is obtained. Next, a first reaction mixture is prepared by combining at least one protein sample with a DNA molecule, comprising a label and DNA-binding sequence, in a well of a first microtiter plate under conditions sufficient to form a binding complex, comprising the DNA-binding protein and the DNA-binding sequence. Then, a second reaction mixture is prepared by combining the protein sample of the first reaction with a DNA molecule of the first reaction and at least one chemical sample potentially containing an inhibitor of the DNA-binding protein in the well of a second microtiter plate under conditions sufficient to form the binding complex, wherein the inhibitor prevents the formation of the binding complex. Next, the first and second reaction mixtures are treated in a manner sufficient to degrade unbound DNA molecules. Finally, the amounts of intact DNA of both first and second reaction mixtures are detected, wherein a lower amount of intact DNA of the second reaction mixture indicates an inhibitor.

In one preferred embodiment of the present invention, a DNA is synthesized, for example, by PCR, to incorporate uracil in place of thymine and/or labeled with a molecular tag (e.g. a fluorophore, a radioisotope, or biotin) to allow for the detection of the DNA molecule. Such DNA with uracil in place of thyrnine can be used in above embodiments. DNA with uracil in place of thymine with a label can also be used in such embodiments, and as follows: a protein sample potentially containing an unknown DNA-binding protein of interest is combined with the DNA. Conditions are provided to allow for a binding complex to form. Next, uracil N-glycosylase is combined with the binding complex, which catalyzes the removal of uracil from the unbound DNA, i.e. the DNA which is not in association with a DNA-binding protein. Uracil contained within the region of DNA that is in association with a DNA-binding protein, i.e. the bound DNA, is protected from the activity of uracil DNA N-glycosylase and will not be removed. The removal of a uracil base forms an AP site. Next, the phosphodiester bonds of both strands of the DNA at the AP sites are cleaved upon exposure to heat and/or alkali conditions. Finally, the label of the bound DNA is detected using detection methods known in the art, such as fluorescence, wherein the detected bound DNA indicates a DNA-binding protein. The DNA-binding protein can be characterized using a variety of methods such as immunodetection, mass spectrometry, amino acid sequencing, and enzymatic digestion of the nucleic acid-binding protein. The technique of gel electrophoresis is not required for the detection of the bound DNA molecule.

Throughout this specification and claims, the word “comprise,” or variations such as “comprises” or “comprising,” will be understood to imply the inclusion of a stated integer or group of integers but not the exclusion of any other integer or group of integers.

These and other embodiments are disclosed or are obvious from and encompassed by, the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 is a schematic representing the use of uracil DNA N-glycosylase to detect a DNA-binding protein.

FIG. 2 demonstrates the detection of a DNA-binding protein.

### DETAILED DESCRIPTION

The present invention is directed to the detection and characterization of proteins that bind to nucleic acids. The method of the instant invention is reliable, efficient, rapid, and scalable and is adaptable to high-throughput processing of a plurality of reactions in parallel. Further, the instant invention is compatible with techniques of automation known in the art. Further still, the method can employ but does not require gel electrophoresis techniques, or any type of fractionation technique, to detect nucleic acid-binding proteins.

The following terms shall have the meaning set forth below:

“Unknown nucleic acid-binding protein” refers to a nucleic acid-binding protein whose nucleic acid-binding function has not previously been elucidated or whose function is not well understood in the art. The biological roles for such proteins include but are not limited to transcription, translation, DNA repair, DNA replication, DNA recombination, and RNA metabolism.

“AP lyase” refers to a type of enzyme that catalyzes the cleavage of a phosphodiester bond adjacent to an AP site. There are two separates classes of AP lyases, wherein the enzymes of the first class are formally known as AP lyases and the enzymes of the second class are formally known as AP endonucleases. Although both types of enzymes produce a nick (a cleaved phosphodiester bond on one strand of the DNA) in the DNA at the AP site, their catalytic mechanisms are slightly different. In the first case, AP lyases cleave the phosphodiester bond 3′ to an AP site by a mechanism that cleaves the deoxyribose moiety of the DNA generating a 5′ phophoryl and 3′ end lacking a hydroxyl. In the second case, AP endonucleases hydrolyze the phophodiester bond 5′to an AP site in a manner that leaves a 3′ hydroxyl end and a 5′ phophoryl end. Some AP lyases also have an associated DNA N-glycosylase activity and therefore, can carry out both steps of AP site formation and phosphodiester bond cleavage.

“DNA N-glycosylase” refers to a type of enzyme that catalyzes the removal of the base portion of a chemically modified or incorrectly inserted nucleotide by cleaving the N-glycosidic bond that links the base to the deoxyribose-phosphodiester backbone of the DNA. The term encompasses both DNA N-glycosylases that have only a glycosylase activity, such as uracil DNA N-glycosylase, and DNA N-glycosylases having an associatcd AD lyase activity, such as endonuclease III and formamidopyrimidine glycosylase. Other specific N-glycosylases will become available and known to those of skill in the art. N-glycosylases are sometimes referred to as “glycosidases” and the term “DNA N-glycosylase” is meant to cover “glycosidases” as well.

“AP site” refers to an apurinic/apyrimidinic site of a DNA molecule that results from treatment with a DNA N-glycosylase. An AP site is one wherein the base portion has been removed from the nucleotide by cleaving the N-glycosidic bond that joins the base with the sugar-phosphate backbone of the DNA.

“Single-base lesion” refers to the state of a DNA molecule following treatment with an AP lyase. Specifically, a single-base lesion represents a cleaved phosphodiester bond on either the 5′ or 3′ side or both sides of an AP site in combination with an AP site.

“Nucleic acid degradation conditions” refers to a treatment of a nucleic acid molecule that involves an enzymatic process or an enzymatic process in combination with a physical (e.g. heat and/or alkali treatment) process that results in the cleavage of one or more phosphodiester bonds of the nucleic acid. Such treatment can be controlled to induce cleavage of a nucleic acid molecule into fragments as well as to hydrolyze a nucleic acid molecule into free nucleotides. The nucleic acid can be RNA or DNA.

“Binding complex” refers to a direct physical association between two macromolecules. In the case of the instant invention, a binding complex encompasses a direct physical association between at least one protein and a nucleic acid molecule. The physical association can comprise “specific-binding”, wherein the protein recognizes and interacts with a defined nucleotide sequence, or “non-specific binding”, wherein the protein does not require a defined nucleotide sequence to associate with the nucleic acid molecule (e.g. a protein that interacts with the phosphate-sugar backbone of the DNA but not the bases of the nucleotides). The strength of the association between the protein and the nucleic acid molecule can vary significantly between different binding complexes. As such, binding complexes can comprise both weakly-bound protein and nucleic acids and strongly-bound proteins and nucleic acids. The strength of the association can be measured, for example, by the stringency of the hybridization conditions or the dissociation constant (KD) of the complex.

“Conditions sufficient to form a binding complex” refers to the physical parameters selected for a binding reaction between a nucleic acid molecule and a protein sample that potentially contains an unknown nucleic acid-binding protein, such as, buffer ionic strength, buffer pH, temperature, incubation time, and the concentrations of nucleic acid and protein. Such conditions can be “low-stringency conditions”, which are conducive to the formation of binding complexes comprising both weakly- and strongly-bound proteins and nucleic acids or “high-stringency conditions”, which are conducive to the formation of binding complexes comprising only strongly-bound proteins and nucleic acids. Low-stringency conditions typically comprise high salt concentration and a temperature ranging between 37C and 47C. High-stringency conditions typically comprise lower salt concentrations, a temperature of 65C or greater, and a detergent, such as sodium dodecylsulfate (SDS) at a concentration ranging from about 0.1% to about 2%.

“Nucleic acid-binding sequence” refers to the region of a nucleic acid molecule, such as a specific DNA sequence, that forms an association with (i.e. binds with) a nucleic acid-binding protein. The nucleic acid molecule can be RNA or DNA.

“Nucleic acid protection” refers to the ability of one or more nucleic acid-binding proteins to prevent enzymatic degradation of the nucleic acid molecule in the region that is in association with the one or more nucleic acid-binding proteins, i.e. the nucleic acid-binding sequence. The nucleic acid can be DNA or RNA.

“Bound nucleic acid” refers to the nucleic acid sequence that is in association with (i.e. bound to) a nucleic acid-binding protein. More specifically, it refers to the portion of a nucleic acid molecule that is protected from enzymatic degradation, i.e. the nucleic acid comprising the nucleic acid-binding sequence. Further, it is the portion of the nucleic acid that is not degraded. The nucleic acid can be DNA or RNA.

“Unbound nucleic acid” refers to the nucleic acid sequence that is not in association with (i.e. not bound to) a nucleic acid-binding protein. More specifically, it refers to the portion of a nucleic acid molecule that is not protected from enzymatic degradation, i.e. the nucleic acid not comprising the nucleic acid-binding sequence. Further, it is the portion of the nucleic acid that is degraded. The nucleic acid can be DNA or RNA.

“Label” refers to a covalently attached molecule to nucleic acid wherein detecting the presence of the label indicates the presence of a nucleic acid molecule. Further, the relative amount of the label that is detected corresponds proportionally to a quantity of nucleic acid. Known labels in the art can be biotin, fluorophores, or radioactive isotopes, such as ³²P, ³³P, or ³⁵S, however, any label known in the art can be employed.

“Detection system” refers to any technique known in the art for detecting a label, such as detection of a fluorophore by light emission (e.g. fluorescence), radioactivity detection of a radioactive isotope emitting, for example, gamma or beta waves, detection of a biotin-labeled nucleic acid by streptavidin conjugated to a colorometric marker, such as LacZ, or more preferably, horseradish peroxidase, and enzyme-linked detection, such as an assayable enzyme covalently attached to an anti-fluorophore or anti-biotin antibody.

“Chemical modification enabling degradation” refers to the prerequisite chemical state of a nucleic acid enabling it to become a substrate for various DNA repair enzymes such as DNA N-glycosylases and AP lyases. Specifically, the chemical modification relates to a chemically modified base or incorrectly inserted nucleotide in the DNA. For example, the enzyme, formamidopyrimidine DNA N-glycosylase, recognizes and removes base modifications including 8-hydroxyguanine, a modified guanine base, and imidazole ring-opened derivatives of adeneine (4,6-diamino-5-formamidopyrimidine) or guanine (2,6-diamino-4-hydroxy-5-formamidopyrimidine). Another example is the enzyme, uracil DNA N-glycosylase, which recognizes and removes uracil that either was misincorporated into the DNA during synthesis or formed by the deamination of cytosine.

“Nucleotide” refers to a base-sugarphosphate compound. Nucleotides are the monomeric subunits of both types of nucleic acid molecules, RNA and DNA. Nucleotide refers to ribonucleoside triphophates, rATP, rGTP, rUTP and rCTP, and deoxyribonucleoside triphosphates, such as dATP, dGTP, dTTP, and dCTP.

“Base” refers to the nitrogen-containing base of a nucleotide, for example adenine (A), cytidine (C), guanine (G), thymine (T), and uracil (U).

“Incorporation” refers to becoming a part of a nucleic acid molecule during synthesis.

The present invention is a reliable, efficient, rapid, and scalable method to detect an unknown DNA- or RNA-binding protein from a biological sample. The method is adaptable to processing a plurality of reactions in parallel and is compatible with techniques of automation known in the art. Further still, the method can employ but does not require gel electrophoresis techniques, or any type of fractionation technique, to detect nucleic acid-binding proteins.

The embodiments of the present invention relate to a novel method for the detection and characterization of one or more DNA- or RNA-binding proteins from a sample. More particularly, the present invention relates to a novel method for the detection and characterization of one or more unknown DNA- or RNA-binding proteins from a biological sample.

The method of the instant invention is generally carried out by first contacting a protein sample potentially containing an unknown nucleic acid-binding protein with a nucleic acid molecule containing at least one nucleic acid-binding sequence under conditions that allow formation of a binding complex between any nucleic acid-binding protein and the nucleic acid-binding sequence of the protein such that the binding complex protects the nucleic acid from degradation. Next, the binding complex is subjected to nucleic acid degradation conditions such that the unbound nucleic acid is degraded but the bound nucleic acid is not degraded. The bound nucleic acid, which comprises the region of nucleic molecule that is contacted by the nucleic acid-binding protein, is protected from degradation and remains intact. The bound nucleic acid is detected by a detection system known in the art. The detection of bound nucleic acid, which represents the intact nucleic acid, indicates the presence of a nucleic acid-binding protein. The instant invention, can employ but does not require gel electrophoresis or any similar type of resolution or fractionation technique to detect the bound nucleic acid. Once the bound nucleic acid is detected, the associated nucleic acid-binding protein can be characterized using any technique known to the skilled artisan for protein characterization.

The protein sample of the instant invention can be of any type known in the art, such as, a cell extract, a partially purified protein solution, or a purified protein solution. The cell extract can be prepared from prokaryotic cells, for example, Gram positive bacteria, Gram negative bacteria, pathogenic bacteria, non-pathogenic bacteria, genome-sequenced bacteria, and non-genome sequenced bacteria, or eukaryotic cells, for example, tissue, blood, fungal cells, protist cells, mammalian cells, and human cells. The invention further contemplates that the protein sample can be prepared from viruses, viral particles, or viral-infected cells. The protein samples can comprise any fraction or sub-fraction of a cell extract, such as, the soluble fraction. The protein sample potentially contains at least one naturally-occurring or one recombinant nucleic acid-binding protein. The nucleic acid-binding protein can be a DNA- or RNA-binding protein. Methods for preparing cell extracts from any type of cell are well-known in the art and can be found in, for example, Sambrook et al., Molecular Cloning: A Laboratory Manual, Cold Spring Harbor Laboratory Press (2001).

The instant invention provides a method to detect both naturally-occurring nucleic acid-binding proteins or recombinant nucleic acid-binding proteins from a protein sample. Nucleic acid-binding proteins can include, but are not limited to, a zinc-finger protein, a helix-turn-helix protein, a eukaryotic transcription factor, a prokaryotic transcription factor, a leucine zipper DNA-binding protein, a beta-domain DNA binding protein, a DNA repair protein, a DNA replication protein, a DNA recombination protein, a ribonucleotide particle protein, a transcription protein, or an RNA-splicing protein.

The nucleic acid molecule of the instant invention can be RNA or DNA. Further, the nucleic acid molecule can comprise a nucleic acid-binding sequence.

For the embodiments that concern a DNA molecule, the invention contemplates that the DNA molecule comprises a DNA-binding sequence and can be any type known in the art, such as, a cDNA, a PCR product, a DNA restriction fragment, a recombinant molecule, a genomic or partial genomic library, a cDNA library, a synthetic DNA and combinations thereof. Methods for preparing or synthesizing all types of DNA molecules are well known in the art and can be found in, for example, Rapley et al. editors, Molecular Biomethods Handbook, Humana Press Inc. (1998).

In a preferred embodiment, the DNA molecule is prepared by PCR and contains a chemical modification that enables the DNA molecule to be degraded by an enzyme, such as, an DNA N-glycosylase or an AP lyase. The DNA N-glycosylase can be any such enzyme known in the art, such as uracil DNA N-glycosylase, 3-methyladenine DNA glycosylase I, 3-methyladenine DNA glycosylase II, pyrimidine hydrate DNA glycosylase (i.e. endonuclease III), formamidopyrimidine (FaPy) DNA glycosylase, or thymine DNA glycosylase. The AP lyase can be any known by the skilled artisan, such as, pyrimidine hydrate DNA glycosylase (endonuclease III), formamidopyrimidine (FaPy) DNA glycosylase, exonuclease III and endonuclease IV. Preferably, the AP lyase further comprises a DNA N-glycosylase activity. Methods of carrying out PCR are well-known in the art and can be found in, for example, Rapley et al. editors, Molecular Biomethods Handbook, Humana Press Inc. (1998) and Dieffenbach et al. editors, PCR Primer: A Laboratory Manual Cold Spring Harbor Press (1998).

In a further preferred embodiment, a DNA molecule comprising at least one DNA-binding sequence is prepared by PCR wherein one or more uracil is incorporated into the molecule in place of one or more thymine, enabling the DNA molecule to be degraded according to the following DNA degradation conditions: the DNA molecule of a binding complex is contacted with the uracil DNA N-glycosylase whereby the uracil DNA N-glycosylase excises the uracil bases of the unprotected (i.e. unbound) DNA molecule forming AP sites, after which the DNA molecule is broken at each AP site following exposure to heat and alkali conditions. In this preferred embodiment, the preferred minimal components for synthesis of the DNA molecule by PCR are: a template DNA comprising at least one DNA-binding sequence (double- or single-stranded), a primer oligodeoxynucleotide of a sequence which is the reverse complement of a portion of the template, the nucleotides adenine, uracil, guanine, and cytosine at a concentration of around 50-250 micromolar, a buffer of pH 6.5-9.0, magnesium ions at 1-10 mM, and a thermostable DNA polymerase enzyme.

In general, methods of synthesizing DNA molecules by PCR are well-known in the art and can be found in, for example, Dieffenbach et al. editors, PCR Primer: A Laboratory Manual, Cold Spring Harbor Press (1998).

In one preferred embodiment, the nucleic acid molecule contains a label. Nucleic acid molecules of the present invention can be either DNA or RNA. More particularly, the nucleic acid molecules can be MRNA, cDNA, PCR products, DNA restriction fragments, recombinant molecules, genomic or partial genomic libraries, cDNA libraries, synthetic DNAs, synthetic RNAs, and combinations thereof. The label can be, but is not limited to, biotin, a radioisotope, such as, ³²P, ³³P, ³⁵S, a fluorohore, such as, fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY. Fluorophores can be obtained commercially from molecular biology supply companies, such as, Molecular Probes (Eugene, Oreg.).

Methods of direct labeling of nucleic acids with fluorophores and enzymatic incorporation of fluorophores into nucleic acids during synthesis are well-known in the art. For example, the ULYSIS Nucleic Acid Labeling Kit (Molecular Probes) can be used to directly label nucleic acids with fluorophores. In another example, ChromaTide Nucleotides (Molecular Probes), which are a series of uridine triphosphates conjugated to a fluorophore, that is enzymatically incorporated into the nucleic acid during synthesis.

Additional methods for labeling nucleic acids can be found in commercially-available molecular biology protocol handbooks, such as, Kricka, “Non-Isotopic Probing, Blotting, and Sequencing,” Academic Press (1995) and Igloi, “Strategies for Introducing Non-Radioactive Labels During the Automated Sequence Analysis of Nucleic Acids,” EJB Electronic Journal of Biotechnology 1:1-8 (1998).

Detecting the label of the nucleic acid molecule can be achieved by any detection system known to a skilled artisan, for example, a radioactivity detection system, a fluorescence detection system, or an enzyme-linked detection system. Radioactivity detection systems are well-known in the art and can include, for example, x-ray film. Methods for detecting radioactivity can be found in published manuals known in the art, for example, Sambrook et al. For embodiments relating to the use of reaction vessels comprising a plurality of wells, such as microtiter plates, the fluorescence detection system can include, for example, a fluorescence multiwell microtiter plate reader, such as the FluorChem 8800 Fluorescence and Chemiluminescence Imaging System (Alpha Innotech Corporation, Burlingame, Calif.), the Aminco-Bowman (Spectronic Instruments, Rochester, N.Y.) Series 2 spectrofluorometer, or the CytoFluor® Fluorescence Multi-Well Plate Reader (Applied Biosystems, Foster City, Calif.). Methods for detecting the label of a nucleic acid using enzyme-linked detection systems are also well-known in the art.

In general, fluorescence detection methods rely on one of the following phenomena: a) a change in the fluorescence intensity of a fluorophore conjugated to a DNA or a protein that occurs upon the formation of a DNA/protein binding complex; b) a change in the polarization of the fluorescence signal of the labeled DNA or protein resulting from the influence on the greater molecular weight of the DNA/protein binding complex; and c) a transfer of energy from one fluorophore on a DNA (or protein) to another fluorophore on a protein (or DNA) resulting in a detectable emission. The fluorescence-based methods mentioned above for detecting DNA-binding proteins in solution are well-known in the art and are described, for example, in Heyduk et al. (Nature Biotechnology 20: 171-177; 2002).

For example, Biotin ChomaTide Nucleotides (Molecular Probes) could be used to incorporate biotin into a DNA molecule during PCR synthesis. The biotin can then be detected using a binding partner, such as, avidin, streptavidin, or anti-biotin antibodies conjugated to an enzyme, such as, alkaline phosphatase (AP). A chromagenic or chemiluminescent substrate of AP, such as, CDP-Star® or CSPD® Chemiluminescent Substrates (Applied Biosystems) can be used to produce a colorometric or fluorescence signal that can be detected. In a preferred embodiment of the instant invention, the binding partner, such as avidin, streptidin, or anti-biotin antibody, is conjugated to a fluorophore, such as, fluorescein, fluorescein-derivative, rhodamine, rhodaminederivative, Texas Red, Oregon Green, Alexa Fluor, Cascade Blue, Tetramethylrhodamine, Pacific Blue, SYBR, and BODIPY.

In yet another preferred embodiment, the nucleic acid molecule is detected by contacting the nucleic acid with nucleic acid dyes, including but not limited to, cyanine, cyanine-derivatives, PicoGreen, OliGreen, RiboGreen, TOTO dyes, intercalating dyes, ethidium bromide, propridium iodide, hexidium idodide, acridine orange, minor-groove-binding dyes, Hoeschst, and DAPI, and then detecting the nucleic acid dye. Nucleic acid dyes are well-known in the art and are commercially available (e.g. Molecular Probes). Methods for detecting nucleic acid dyes are well-known in the art. Ethidium bromide, for example, is visualized under ultraviolet light. Other methods of using nucleic acid dyes can be found in Sambrook et al.

The method of the instant invention further involves forming a binding complex, comprising a bound nucleic acid molecule and a nucleic acid-binding protein, wherein said binding complex protects the bound nucleic acid molecule from degradation. The nucleic acid may be DNA or RNA. Methods for forming binding complexes between nucleic acids and protein are well-known in the art and can be found in commercially-available protocol handbooks, for example, Sambrook et al. and Tymms “Transcription Factor Protocols,” Humana Press, volume 131 Methods in Molecular Biology Series, (2000).

In a preferred embodiment, conditions sufficient to form a binding complex comprise preparing a mixture comprising 20 mM Tris-Cl pH 8.0, 80 mM KCl, 1 mM EDTA, 1 mM DTT, 12.0% glycerol, 0.05 μg/ml of salmon sperm DNA, 300 μg/ml of Bovine serum albumin, the nucleic acid molecule, and the protein sample and incubating the mixture for 30 minutes at 30° C. In a further preferred embodiment, the binding complexes are allowed to form in the wells of reaction vessel comprising a plurality of wells, such as, a microtiter plate. In the case of a microtiter plate, it can have any array-format known in the art, such as, a 96- or 384-well format. Further, the wells of the microtiter plates can be coated with streptavidin for the capturing (binding) of biotin-labeled nucleic acid molecules or proteins.

Microtiter plates and streptavidin-coated microtiter plates are available commercially from molecular biology supply companies, such as, Thermo Labsystems USA (Franklin, Mass.), Pierce Biotechnology, Inc. (Rockland, Ill.), and Sigma-Aldrich Corp. (St. Louis, Mo.). Nucleic acid degradation conditions can be selected to suit particular embodiments of the invention. In one preferred embodiment, unbound nucleic acid degradation conditions are enzymatic. In embodiments involving DNA that is free of any chemical modifications, the enzyme can be, for example, a DNase, such as DNase I. The DNA can be degraded by contacting the binding complex with the DNase, for example, DNase I, in an amount sufficient to degrade unbound DNA molecules.

Methods of using a DNase are well-known in the art. In embodiments involving RNA, the enzyme can be, for example, an RNase, such as, RNase A, RNase H, or RNase T. Likewise, the RNA can be degraded by contacting the binding complex with the RNase in an amount sufficient to degrade unbound RNA molecules. Methods of using RNases are well known by one of skill in the art.

In another preferred embodiment, the nucleic acid degradation conditions are both enzymatic and physical. The invention contemplates using a DNA N-glycosylase to form an AP site, wherein the AP site is cleaved by exposing the nucleic acid to heat and alkali conditions. For example, the following method can be carried out to degrade the nucleic acid using enzymatic and physical conditions: Nucleic acids containing AP sites following removal of a chemically modified or misincorporated base, such as uracil, are heated in a buffer solution containing an amine, for example, 25 mM Tris-HCl and 1 to 5 mM magnesium ions, for a period of 10 to 30 minutes at 70° C. to 95° C. Alternatively, the following treatment can be used to break the DNA at AP sites: 1.0 M piperidine, a base, is added to DNA which has been precipitated with ethanol and vacuum dried. The solution is then heated for 30 minutes at 90° C. and lyophilized to remove the piperidine.

Embodiments involving the formation of an AP site in DNA using an enzyme, such as, an DNA N-glycosylase, for example, uracil DNA N-glycosylase, comprise introducing a chemical modification in the nucleotide base of one or more guanine, cytosine, thymine, or adenosine, enabling degradation by enzymes. Advantageously, the chemical modification is introduced prior to the treatment of the DNA by the enzyme. The chemical modification creates a prerequisite chemical state in a nucleic acid enabling it to become a substrate for various DNA repair enzymes, such as DNA N-glycosylases and AP lyases. The chemical modification comprises a chemically modified base or incorrectly inserted nucleotide in the DNA. For example, formamidopyrimidine DNA N-glycosylase recognizes and removes base modifications including 8-hydroxyguanine, a modified guanine base, and imidazole ring-opened derivatives of adeneine (4,6-diamino5-formamidopyrimidine) or guanine (2,6-diamino-4-hydroxy-5-forrnamidopyrimidine).

Another example is uracil DNA N-glycosylase, which recognizes and removes uracil that was either incorporated into the DNA during synthesis or formed by the deamination of cytosine. Degradation of the DNA molecule according to the present embodiment comprises the steps of contacting the DNA molecule with the DNA N-glycosylase, excising one or more nucleotide bases of the DNA molecule having the chemical modification, forming an AP site at each excises nucleotide base, and exposing the DNA molecule to heat and alkali condition to cause a break in the DNA molecule at each AP site.

In yet another advantageous embodiment, the enzyme that degrades the unbound DNA molecule is both a DNA N-glycosylase and an AP lyase. The DNA N-glycosylase can include, but is not limited to, uracil DNA N-glycosylase, 3-methyladenine DNA glycosylase I, 3-methyladenine DNA glycosylase II, pyrimidine hydrate DNA glycosylase (endonuclease III), formamidopyrimidine (FaPy) DNA glycosylase, thymine mismatch DNA glycosylase or combinations thereof. The AP lyase can include, but is not limited to, pyrimidine hydrate DNA glycosylase (endonuclease III), formamidopyriminde (FaPy) DNA glycosylase, exonuclease III, endonuclease IV or combinations thereof.

According to this embodiment, the degradation of a DNA molecule comprises the steps of contacting the DNA molecule with a DNA N-glycosylase to introduce a chemical modification, excising one or more nucleotide bases of the DNA molecule having the chemical modification, forming an AP site at each excised nucleotide base, contacting the DNA molecule with AP lyase, forming a single-base lesion at each AP site, and exposing the DNA molecule to heat and alkali conditions to cause a break in the DNA molecule at each single-base lesion. The DNA N-glycosylase and the AP lyase can comprise the same enzyme, e.g. one enzyme may possess both activities, such as with pyrimidine hydrate DNA glycosylase (endonuclease III) and formamidopyriminde (FaPy) DNA glycosylase.

In yet another preferred embodiment, the nucleic acid-binding protein is identified and/or further characterized. The nucleic acid can be DNA or RNA. In one preferred embodiment, the step of characterizing the nucleic acid-binding protein comprises the technique of mass spectrometry. The technique of mass spectrometry generally comprises introducing the binding complex to a mass spectrometer, forming characteristic fragment ions of the nucleic acid-binding protein, and identifying the protein by comparing the characteristic fragment ions with standards. Methods of mass spectrometry can be found in the scientific literature available in the art, such as, Grant et al., “Proteomics of Multiprotein Complexes: Answering Fundamental Questions in Neuroscience,” Trends in Biotechnology, 19(10 Suppl):S49-54 (2001) and Simpson et al., “Cancer Proteomics: From Signaling Networks to Tumor Markers,” Trends in Biotechnology (2001).

In another preferred embodiment, the step of identifying and/or further characterizing the nucleic acid-binding protein comprises the technique of amino acid sequencing. The technique of amino acid sequencing comprises a method for the characterization of the linear sequence of amino acid residues of a protein or a protein fragment from one end, for example, the amino-terminal end. Methods for amino acid sequencing can be found in commercially-available molecular biology or biochemistry protocol manuals, such as, Walker, The Protein Protocols Handbook, 2^{nd} Edition, Humana Press (2002).

In yet another preferred embodiment, the step of identifying and/or characterizing the nucleic acid-binding protein comprises the technique of enzymatic digestion. Methods for enzymatic digestion of proteins can be found in commercially-available molecular biology or biochemistry protocol manuals, such as, Walker, The Protein Protocols Handbook, 2^{nd} Edition, Humana Press (1996).

In still yet another preferred embodiment, the step of identifying the nucleic acid-binding protein comprises the technique of immunodetection. Immunodetection comprises the steps of contacting antibodies raised against known nucleic acid-binding protein with the nucleic acid-binding protein under conditions sufficient to form a protein-antibody complex, detecting the protein-antibody complex, and identifying and/or characterizing the nucleic acid-protein of the protein-antibody complex. Antibodies raised against known nucleic acid-binding proteins are available commercially and can be obtained by the skilled artisan. Methods of immunodetection can be found in commercially-available molecular biology and protein science protocol manuals, such as, Delves, Antibody Applications: Essential Techniques, John Wiley & Sons, Ltd., (1995). In one embodiment, the antibody of the protein-antibody complex can be covalently coupled to a secondary detectable protein or molecule, such as an enzyme that can be assayed (e.g. horseradish peroxidase or alkaline phosphatase), a fluorescent protein (e.g. green fluorescent protein), or a fluorophore (e.g. fluoresceine isothiocyanate.) Detection of the antibody can be carried out by detecting the covalently coupled enzyme, fluorescent protein or fluorophore.

In yet another preferred embodiment, the antibody of the protein-antibody complex can be detected through the use of a secondary antibody. The use of a secondary antibody involves contacting a first antibody with a secondary antibody that is raised against the first antibody and is covalently-coupled to a detectable protein or label, such as an enzyme that can be assayed (e.g. horseradish peroxidase or alkaline phosphatase), a fluorescent protein (e.g. green fluorescent protein), or a fluorophore (e.g. fluoresceine isothiocyanate.) Methods for using secondary antibodies in immunodetection techniques is well-known in the art and can be found in commercially-available protocol books, such as Delves, Antibody Applications: Essential Techniques, John Wiley & Sons, Ltd., (1995).

In a further preferred embodiment, the method of the instant invention comprises obtaining one or more protein samples, wherein each protein sample potentially contains a least one unknown DNA-binding protein and combining said protein sample with at least one DNA molecule, comprising a DNA-binding sequence and a label, in one or more wells of a reaction vessel comprising a plurality of wells, such as, a microtiter plate. Next, the DNA molecule and the protein sample in the one or more wells of the reaction vessel are exposed to conditions sufficient to form a binding complex, comprising the DNA-binding protein and a bound DNA molecule, wherein the binding complex protects the bound DNA molecule or fragment thereof from degradation. Then, the binding complex is subjected to DNA degradation conditions, wherein any unbound DNA molecules are degraded. Lastly, any bound DNA is detected, wherein detecting the bound DNA indicates a DNA-binding protein. Preferably, detection is carried out in a hight-throughput manner.

For example, the DNA can be bound to magnetic microparticles in the one or more wells of the reaction vessel comprising a plurality of wells, such as a microtiter plate, to facilitate liquid handling and automation.

In a further preferred embodiment, an inhibitor of a DNA-binding protein can be detected. One or more protein samples potentially containing one or more unknown DNA-binding proteins is obtained. In addition, one or more chemical samples potentially containing one or more inhibitors of the DNA-binding protein is obtained. Next, a first reaction mixture is prepared by combining at least one protein sample with a DNA molecule, comprising a label and DNA-binding sequence, in a first well of a reaction vessel comprising a plurality of wells, such as a microtiter plate, under conditions sufficient to form a binding complex, comprising the DNA-binding protein and the DNA-binding sequence. Then, a second reaction mixture is prepared by combining the protein sample of the first reaction with a DNA molecule of the first reaction and at least one chemical sample in a second well of a reaction vessel comprising a plurality of wells, such as a microtiter plate, under conditions sufficient to form the binding complex, wherein the inhibitor prevents the formation of the binding complex. Next, the first and second reaction mixtures are treated in a manner sufficient to degrade unbound DNA molecules. Finally, the amounts of intact DNA of both first and second reaction mixtures are detected, wherein a lower amount of intact DNA of said second reaction mixture indicates the presence of an inhibitor.

In one preferred embodiment of the present invention, a DNA is synthesized, for example, by PCR, to incorporate uracil in place of thymine and labeled with a molecular tag (e.g. a fluorophore, a radioisotope, or biotin) to allow for the detection of the DNA molecule. Next, a protein sample potentially containing an unknown DNA-binding protein of interest is combined with the DNA. Conditions are provided to allow for a binding complex to form. Next, uracil N-glycosylase is combined with the binding complex, which catalyzes the removal of uracil from the unbound DNA, i.e. the DNA which is not in association with a DNA-binding protein. Uracil contained within the region of DNA that is in association with a DNA-binding protein is protected from the activity of uracil DNA N-glycosylase and will not be removed. The removal of a uracil forms an AP site. Next, the phosphodiester bonds of both strands of the DNA at the AP sites are cleaved upon exposure to heat and alkali conditions. Finally, the label of the bound DNA is detected using a detection method, such as fluorescence, wherein the detected bound DNA indicates a DNA-binding protein. The DNA-binding protein may then be identified using a variety of methods such as a immunodetection, mass spectrometry, sequence analysis, or enzymatic digestion, which are all methods of which are well-known in the art and described previously in the present application. The technique of gel electrophoresis is not required for the detection of the bound DNA molecule.

In embodiments pertaining to the use of microtiter plates, e.g. “microwell plates”, coating technologies known in the art can be utilized to enable the attachment or affixing to the surface of a reaction vessel, such as, for example, the well of a microtiter plate, the macromolecules of the invention, such as, for example, nucleic acid molecules comprising a binding site of a nucleic acid-binding protein. Any coating technology known to one of ordinary skill in the art to attach or affix the nucleic acid molecules and/or proteins of the invention or those coating technologies yet to be developed are contemplated by the present invention.

For example, a coating technology known in the art utilizes the protein streptavidin and its cognate ligand biotin. This particular example of coating technology is useful in binding or affixing a macromolecule of interest to a surface, such as, for example, the surface of a microtiter plate well.

The macromolecule can be for example a nucleic acid molecule such as a PCR-generated DNA fragment or a particular protein or polypeptide. In operation, the macromolecule of interest is labeled with biotin. Consequently, the biotin-labeled macromolecule binds to the surface vis-à-vis the coated streptavidin.

Streptavidin-coated polystyrene microwell plates are readily available in the art (Pierce, Rockland, Ill.) and methods for preparing streptavidin-coated microwell plates will be known by one of ordinary skill in the art. Streptavidin-coated microwell plate wells provide for the specific binding of biotin-labeled nucleic acid fragments or proteins effectively resulting in the binding of the nucleic acid fragments or protein to the surface of the microwell plate well. It will be appreciated that biotin binds strongly to streptavidin and thus any solid support or surface such as a microtiter plate well that is derivatized with the protein streptavidin can be further derivatized by contacting the surface with a molecule comprising a biotin group. The surface materials used can include for example, polystyrene, polyvinyl chloride or polycarbonate microtiter plates or beads and derivatized agarose or acrylamide beads.

In addition to coupling the reaction vessel surface with streptavidin and biotin-DNA (or nucleic acid) complex, the reaction vessel surface can also be derivatized directly with nucleic acid through the use of DNA-interaction promoting materials such as glycine. In addition, esters contained on a reaction vessel surface can be used to react with amine-containing second molecules, such as a nucleic acid derivatized with an amino group at either the 5′ or 3′ end, to produce a reaction surface comprised of covalently coupled second molecule. Further detail on methods of attachment of nucleic acids to surfaces and related chemical processes can be found in U.S. Pat. Nos. 5,667,976, 5,457,025, and 6,268,128, Kelly (2001) Strategies for Attaching Oligonucleotides to Solid Supports, Technical Bulletin of IDT DNA Technologies (http://www.idtdna.com) and Schena (2003) Microarray Analysis, Chapters 5-7, Liley-Liss, each incorporated herein by reference in their entirety.

Nucleic acid may be biotinylated in several ways. DNA can be restricted by an endonuclease which leaves a 5′ overhang serving as a substrate for DNA polymerase. As such, a biotinylated nucleotide, like biotin-16-dUTP (10 mM) can be included in a reaction mix consisting of dCTP, dGTP, and dATP at 1 mM each, magnesium chloride (10 mM), sodium chloride (100 mM), buffer, and Klenow polymeras (1 U). The polymerase incorporates the biotinylated nucleotide into the DNA strand, which allows it to be adsorbed to the solid support. Alternatively, a biotinylated oligonucleotide can be synthesized and used in the polymerase chain reaction (PCR) to produce the biotinylated DNA of interest using one or more nucleotides derivatized with biotin. The resulting biotinylated DNA can be purified by standard chromatography or by other means known in the art such as HPLC and gel purification.

Following adsorption of the DNA to the reaction vessel surface, non-specific protein can be allowed to adsorb to the reaction surface to help minimize background signal. The nonspecific proteins can include, but are not limited to, bovine serum albumin and milk proteins.

In the present invention the insoluble support or substrate will typically be comprised of polymeric material containing derivatizable functional groups [e.g., poly(p-aminostyrene)] or polymeric solid supports that can be activated (e.g., nylon beads, cyanuric chloride activated cellulose commercially available). Examples of preferred insoluble support or substrate compositions include nylon, polystyrene, glass, polypropylenes, polystyrene/glycidyl methacrylate latex beads, latex beads containing amino, carboxyl, sulfonic and/or hydroxyl groups, polystyrene coated magnetic beads containing amino and/or carboxylate groups, glass, teflon, plastic and the like. Also contemplated are metal surfaces (steel, gold, siver, aluminum, silicon and copper), plastic materials including mulitwell plates or membranes comprised of polyethylene, polyproylene, polyamide, or polyvinylenedifluoride and other such materials, wafers, combs, pins (e.g., arrays of pins suitable for combinatorial synthesis or analysis) or beads in pits of flat surfaces such as silicon wafers.

For high throughput assays, the support materials can be comprised of reaction vessels, such as microtiter plate wells, and insoluble supports or substrates, such as glass surfaces, in which high-density arrays of reaction chambers or reaction locations, respectively (e.g., the area or position on a DNA or protein microarray containing the coupled DNA or protein target sample) are possible. The support materials (i.e. the reaction vessels and insoluble substrates) can be derivatized with a macromolecule, such as, for example, DNA, RNA, or protein. Often solid supports derivatized with a protein can be further derivatized with another molecule. For instance, solid supports derivatized with the protein streptavidin can be further derivatized by contacting the solid support with a molecule containing biotin, such as, for example biotin-labeled DNA.

In another aspect, the instant invention relates to novel coating technologies. In one embodiment, amino-poly(A) (adenine) oligonucleotide is conjugated to IgG which is first immobilized onto the surface of the wells of a microtiter plate. The amino-poly(A) oligonucleotide can be linked by a cross-linking reagent, such as, for example, gluteraldehyde. One of ordinary skill in the art will appreciate that in general, crosslinking can be used to form covalent linkages formed between particular functional groups of biomolecules, such as proteins and nucleic acids, to small molecules, such as drugs, toxins, peptides, dyes, haptens, and fluorescent compounds. Further detail on crosslinking, crosslinking reagents, and detection methods can be found in The Handbook of Fluorescent Probes and Research Products, ninth edition, R. Haugland, 2002, which is incorporated in its entirety herein by reference.

In the present embodiment the IgG provides a first layer to which the poly(A) oligonucleotide is linked thereto. The conjugated poly(A) forms an efficient generic capture reagent for hybridizing with any nucleic acid sample, i.e. the poly(A) portion can capture any nucleic acid molecule having a poly(T) tail. As it is referenced in the present application, by “generic capture agent” it is meant a first molecule that is capable of “capturing”, i.e. binding, immobilizing, interacting with, or hybridizing in the case of nucleic acids, any member of heterologous group of second molecules. For example, the poly(A) portion of the poly(A)-IgG conjugate is a generic capture agent for any T-tailed genomic DNA fragment. “T-tailed” or “poly(T) tailed” refers to a DNA molecule that has been coupled to a oligonucleotide successive thymine nucleotides. Methods for T-tailing are known in the art.

Without being bound by theory, the poly(A) generic capture reagent likely introduces less steric hindrance as a result of its conjugation to IgG than if attached directly to the microwell plate surface. Consequently, hybridization between the poly(A)-IgG conjugate poly(T)-tailed nucleic acids is more rapid and efficient as the poly(A) portion is more accessible to the poly(T)-tailed nucleic acids. Further, since the method of the instant invention can be carried out in the microtiter well format, high throughput screening for unknown DNA binding proteins is facilitated. The poly(T) tail can be added or coupled to the oligonucleotide by enzymatic (e.g. vis-à-vis PCR template) or synthetic (e.g DNA synthesizer) processes and is typically attached at the 3′or 5′ ends. Methods for attaching proteins, such as IgG, to microtiter plates are well-known in the art.

In yet another embodiment, the method of the instant invention is carried out using anti-FITC (“fluorescein-5-isothiocyanate”) coated microtiter plates. One of ordinary skill in the art will appreciate that FITC is typically used as a fluorescent reporter molecule for use in detecting macromolecules. For example, oligonucelotide conjugates of FITC can be used as hybridization probes. In the present embodiment, anti-FITC is used to effectively capture nucleic acid molecules that are conjugated or coupled covalently to FITC. Anti-FITC antibodies (Goat-anti-FITC IgG; Rabbit anti-FITC F(Ab)) are available commercially (DAKO, AUSTRALIA). One of ordinary skill in the art will appreciate that that compounds and methods are available for covalently linking or labeling nucleic acids and proteins with FITC (see Handbook of Fluorescent Probes and Research Products, ninth edition, R. Haugland, 2002). Also, it will be appreciated that derivatives of FITC, such as, for example, 6-(fluorescein-5-carboxamido) hexanoic acid and fluorescein-6-isothiocyanate, and derivatives having succinimidyl esters and succinimidyl esters with spacer groups, are also contemplated by the present invention. The FITC-nucleic acid molecule target can be additionally labeled with biotin or other similar and/or analogous reporter molecules. One of ordinary skill in the art will appreciate that the FTC-nucleic acid molecule can be synthesized in the form of an oligonucleotide having a FITC group at the 5′ end.

Also within the scope of the invention are the use of alternate fluorescent labeling reagents and their respective cognate antibodies, such as, for example, Oregon Green (MOLECULAR PROBES, EUGENE, Oreg.) to the extent that the antibody against the particular fluorescent labeling reagent of interest is coupled to the reaction vessel (e.g. the well of a microtiter plate) and the fluorescent labeling reagent is coupled to the nucleic acid molecule of the invention.

In one preferred embodiment of the present invention, a DNA is synthesized, for example, by PCR or DNA synthesis, to incorporate uracil in place of thymine and labeled with a molecular tag (e.g. a fluorophore or a radioisotope) to allow for the detection of the DNA molecule. In addition, the DNA is labeled with a secondary marker, such as biotin, using any known method in the art such as incorporation during synthesis of the DNA molecule or during PCR. Next, a protein sample potentially containing an unknown DNA-binding protein of interest is combined with the DNA in a well of a microtiter plate. One of ordinary skill in the art will readily appreciate that the microtiter plate format enables a multitude of same or different DNA molecules to be used and/or a multitude of protein samples capable of being screened simultaneousiy.

Next, conditions are provided to allow for a binding complex to form between any DNA binding proteins contained in the protein samples and their cognate DNA binding sequences. Next, uracil N-glycosylase is added to each well to catalyze the removal of uracil from any unbound DNA, i.e. the DNA which is not bound to or complexed with a DNA-binding protein. Uracil contained within the region of DNA that is in direct association with a DNA-binding protein is protected from the activity of uracil DNA N-glycosylase and will not be removed. The removal of a uracil forms an AP site. Next, the phosphodiester bonds of both strands of the DNA at the AP sites are cleaved upon exposure to heat and alkali conditions. Finally, the molecular tag of the bound DNA is detected using a detection method, such as fluorescence, wherein the detected bound DNA indicates a DNA-binding protein. Throughout the assay, the intact DNA, i.e. the DNA bound by a DNA-binding protein, remains bound to the streptavidin vis-à-vis the biotin group. The degraded DNA does not remain bound to streptavidin and can be removed through simple washing and rinsing steps.

The DNA-binding protein can then be identified using a variety of methods such as a immunodetection, mass spectrometry, sequence analysis, or enzymatic digestion, which are all methods of which are well-known in the art and described previously in the present application. The technique of gel electrophoresis is not required for the detection of the bound DNA molecule.

In yet another advantageous embodiment of the instant invention, a DNA-binding protein is detected and identified using a “reflex” process comprising the steps of obtaining one or more protein samples, wherein each protein sample potentially contains at least one unknown DNA-binding protein and combining the protein sample with at least one DNA molecule, comprising a DNA-binding sequence and a label, in one or more wells of a reaction vessel comprising a plurality of wells, such as a microtiter plate. Next, the DNA molecule and the protein sample in the reaction vessel are exposed to low-stringency conditions sufficient to form a non-specific binding complex, comprising the DNA-binding protein and a bound DNA molecule, wherein the binding complex protects the bound DNA molecule or fragment thereof from degradation. Thereafter, the reaction mixture is treated in a manner sufficient to degrade unbound DNA molecules. Any bound DNA is detected, wherein detecting the bound DNA indicates a potential DNA-binding protein. Next, protein samples identified as potentially containing a DNA-binding protein are contacted again with the DNA molecule under highly stringent conditions sufficient to form a specific binding complex comprising the DNA-binding protein bound to its preferred DNA-binding sequence, wherein the binding complex protects the bound DNA molecule or fragment thereof from degradation. Then, the reaction mixture is treated in a manner sufficient to degrade unbound DNA molecules. The reflex process can be repeated under conditions of progressively increasing stringency carried out in a high-throughput manner, for example, wherein the DNA is bound to magnetic microparticles in the wells of the reaction vessel, such as microtiter plate, to facilitate liquid handling and automation.

In yet another preferred embodiment, the first phase of the reflex process, employing low-stringency conditions, is applied to a large pool of potential nucleic acid-binding proteins and the second phase of the reflex process, employing high-stringency conditions, is applied to a subset of that pool.

In yet another preferred embodiment, nucleic acid molecules are coupled to microparticles to facilitate different aspects of the method of the instant invention, such as, liquid handling and automation. The microparticles can be coated with antibodies, streptavidin, avidin, biotin, protein, intercalating dyes, or combinations thereof. In a preferred embodiment, the microparticles comprise magnetic microparticles or paramagnetic microparticles wherein the nucleic acid molecules of interest are bound to the magnetic microparticles in the wells of a reaction vessel comprising a plurality of wells, such as a microtiter plate. The methods of the instant invention can be practiced using any silica magnetic particle. Preferably, the methods are practiced using siliceous-oxide coated magnetic (SOCM) particles.

Commercial magnetic microparticles and reaction vessels comprising a plurality of wells, such as microtiter plates, are well-known in the art and can be readily obtained. For example, microtiter plates can be obtained from Thermo Labsystems USA (Franklin, Mass.), Pierce Biotechnology, Inc. (Rockland, Ill.), and Sigma-Aldrich Corp. (St. Louis, Mo.). Reaction vessels can also be coated with streptavidin or avidin to capture nucleic acid molecules and/or proteins coupled to biotin. Microparticles, such as magnetic microparticles, can be obtained from molecular biology supply companies, such as Dynal Biotech, Inc. (Lake Success, N.Y.), Seradyn (Indianapolis, Ind.), and Sperotech, Inc. (Indianapolis, Ill.). Methods for producing magnetic microparticles are also known in the art.

A most preferred method for producing magnetic microparticles for use in the present invention comprises the general steps of: (1) preparing magnetite core particles by aqueous precipitation of a mixture of FeCl₂ and FeCl₃; (2) depositing a siliceous oxide coating on the magnetite core particles by exposing a slurry of the particles to a mixture of SiO₂ and Na₂O for at least about 45 minutes at a temperature of at least about 60° C. and then adding an acid solution to the mixture until the pH is lowered to a pH less than 9; (3) allowing the resulting slurry to age for at least about 15 minutes, preferably while continuing to agitate the slurry; and (4) washing the particles. The deposition and aging steps of the preferred particle production method described above can be repeated to produce multiple layers of siliceous oxide coating over the magnetite core, thus providing additional insurance against leaching of metals from the core into the surrounding environment. Magnetic microparticles produced by the method described above are most preferably treated by being subjected to a mild oxidizing step to further inhibit leaching from the core.

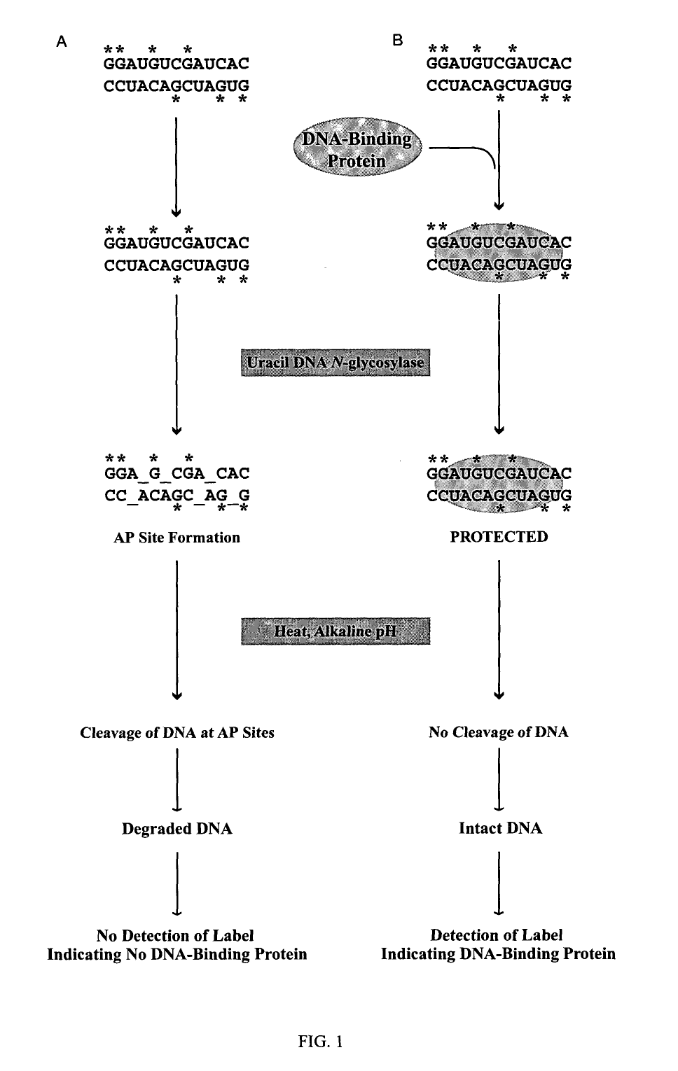

FIG. 1 depicts a schematic that outlines an embodiment of the method of the instant invention. The figure compares two scenarios. In the first scenario (FIG. 1A), a 13 base-pair double-stranded DNA molecule containing a DNA-binding sequence is shown, wherein each guanine nucleotide carries a label (indicated by asterisks). Further, the DNA molecule was prepared by incorporating uracil nucleotides in place of thymine nucleotides. Methods known in the art, such as PCR or DNA chemical synthesis, could be used to prepare the DNA molecule having incorporated uracil. The label could be any known in the art, for example, a fluorophore.

In the second scenario (FIG. 1B), the identical DNA molecule is prepared, however, it is additionally contacted with a DNA-binding protein which is allowed to form a protein-DNA complex with the DNA molecule. Next, both DNA molecules in FIGS. 1A and 1B are exposed to uracil DNA DNA-gycosylase, which catalyzes the removal of each accessible uracil base of each uracil nucleotide by cleaving the N-glycosidic bond attaching the base to the sugar-phosphate backbone of the nucleotide. The removal of the uracil base forms an AP site.

However, in the case of FIG. 1B, the binding of the DNA-binding protein blocks the activity of the uracil DNA N-glycosylase thereby “protecting” the DNA molecule. Next, both DNA molecules of FIGS. 1A and 1B are exposed to heat and alkaline pH, which causes the DNA to be cleaved at each AP site. Since there are no AP sites in the protected DNA molecule, it remains intact. However, the unprotected DNA molecule, which contains multiple AP sites, is degraded upon exposure to heat and alkaline pH. Finally, the label of the protected DNA molecule is detected, which indicates the presence of a DNA-binding protein.

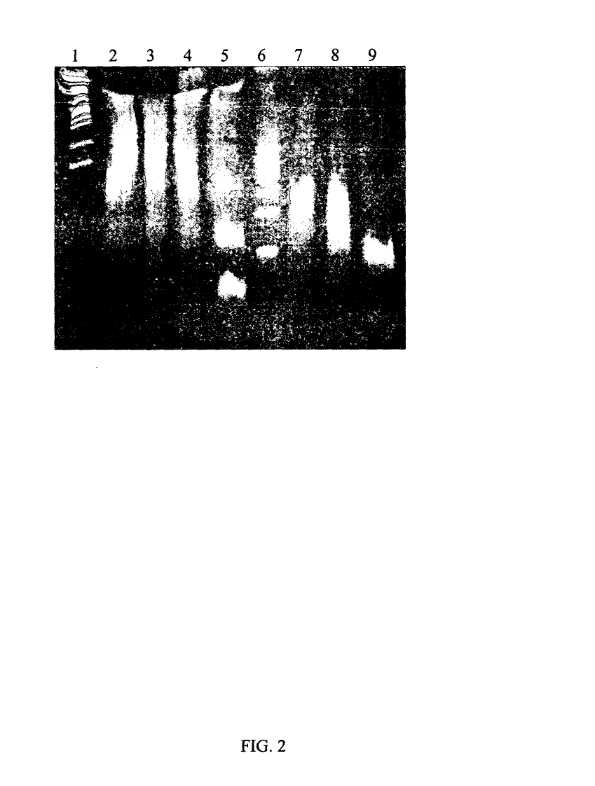

FIG. 2 shows the results of polyacrylamide gel electrophoresis of DNA fragments demonstrating the utility of the instant. The description of this Figure corresponds to Example 1 of the instant application.

The method of the instant invention has a number of advantages. A principle advantage is the ability to detect unknown nucleic acid-binding proteins in a manner that is faster, more efficient, more reliable, and easier to use than current technologies.

A further advantage of the instant invention is that it can be used to detect a plurality of nucleic acid-binding proteins from a plurality of protein samples in parallel. In other words, the instant invention is amendable to being carried out in a high-throughput manner. As discussed previously, methods of the instant invention can utilize reaction vessels comprising a plurality of wells, such as microtiter plates, to facilitate parallel processing of a multitude of protein samples. Advantageously, each step of the method can be carried out using the same microtiter plate, including the final step of detecting the nucleic acid-binding protein. Current technologies are limited by the number of protein samples which can feasibly be screened at any given time since their methods are more cumbersome and labor-intensive that the methods of the current invention.

Another advantage of the invention over current technologies is the ability to quickly and efficiently validate the detection of unknown nucleic acid-binding proteins by carrying out successive rounds of the instant method wherein the stringency conditions for binding complex formation are progressively increased (i.e. the reflex process). At the end of the first phase, potential nucleic acid-binding proteins are detected. A subset of the protein samples containing the unknown nucleic acid-binding proteins are subsequently validated by repeating the methods under conditions of progressively increasing stringency.

A better understanding of the present invention and of its many advantages will be had from the following examples which further describe the present invention and given by way of illustration. The examples that follow are not to be construed as limiting the scope of the invention in any manner. In light of the present disclosure, numerous embodiments within the scope of the claims will be apparent to those of ordinary skill in the art.

### EXAMPLES

### Example 1

Use of Uracil N-Glycosylase in Detecting a DNA-Binding Protein

Uracil N-glycosylase (Invitrogen Corp.) was used in a macromolecular protection assay to demonstrate detection of a DNA-binding protein using the methods of the present invention. The assay employed a control protein, Human AP-2, which is a 52 kDa eukaryotic transcription factor, and the cognate DNA-binding sequence of AP-2.

Four different 38 bp DNA fragments were synthesized containing the DNA-binding sequence for the DNA-binding protein AP-2. The first DNA fragment was prepared as normal DNA (indicated as AP2). The second DNA fragment was prepared with uracil incorporated in place of thymine in one location in the AP-2 binding site on one strand of the DNA fragment (AuP2), the third DNA fragment was prepared with uracil incorporated in place of thymine in one location in the AP-2 binding site on the alternate strand of the DNA fragment (APu2). The fourth DNA fragment was prepared with uracil incorporated in place of thymine in one location in the AP-2 binding site on both strands of the DNA fragment (indicated as AuPu2). The sequences are as follows:

AP2:
5′AAGCTTGATCGAACTGACCGCCCGCGGCCCGTGGATCC 3′
3′TTCGAACTAGCTTGACTGGCGGGCGCCGGGCACCTAGG 5′
AuP2:
5′AAGCUUGAUCGAACUGACCGCCCGCGGCCCGUGGAUCC 3′
3′TTCGAACTAGCTTGACUGGCGGGCGCCGGGCACCTAGG 5′
APu2:
5′AAGCTTGATCGAACTGACCGCCCGCGGCCCGTGGATCC 3′
3′UUCGAACUAGCUUGACUGGCGGGCGCCGGGCACCUAGG 5′
AuPu2:
5′AAGCUUGAUCGAACUGACCGCCCGCGGCCCGUGGAUCC 3′
3′UUCGAACUAGCUUGACUGGCGGGCGCCGGGCACCUAGG 5′

Two sets of reactions were carried out for each DNA fragment. In the first set of reactions, uracil DNA N-glycosylase was contacted with the DNA fragments to catalyze the removal of any uracils from the DNA creating AP sites, followed by exposure to heat and alkaline pH to cleave the DNA at any AP sites. In the second set of reactions, the DNA fragments were first allowed to contact a cell extract containing the AP-2 protein for 30 minutes at room temperature, prior to contacting with uracil DNA N-glycosylase.

Each reaction was performed in a buffer comprising 2 ul of 5× enzyme buffer (100 mM Tris pH 8.0; 250 mM KCl; 25 mM Mg₂Cl), 6 ul of a binding reaction mixture, and 1 ul of water. Reactions were allowed to proceed for 1 hour at 37 ° C.

Cleavage of UNG-digested DNA was facilitated by the addition of 10 μL of NH₄OH and the exposure to heat (65° C. for 1 hour), which cleaves the DNA at each AP site. The digestion products were fractionated on a polyacrylamide gel (20%; 29:1 bis:acrylamide) and visualized by ethidium bromide staining.

The results showed that the uracil-incorporated DNA fragments were sensitive to the UNG degradation process using following treatment with UNG plus exposure to heat and alkaline pH. This was evidenced by two distinctly-sized DNA fragments of test DNA produced by cleavage at the sites of uracil incorporation in the absence of AP-2 (FIG. 2, lane 5). However, in the presence of AP-2, the 38 bp test DNA remained intact as a single polynucleotide (FIG. 2, lane 9). In this case, the uracil-containing sites were “protected” from UNG digestion and heat/alkali cleavage; thus, the 38 bp fragment remained intact. The control DNA was not susceptible to degradation by UNG whether in the presence or absence of AP-2 since no uracil was incorporated into the sequence (FIG. 2, lanes 2 and 7).

This experiment showed that UNG can be used to detect a DNA-binding protein through the analysis of the degradation state of the DNA molecule. In this case, the degradation state of the molecule related to the formation of cleavage products in the absence of a DNA-binding protein.

This demonstrates that the cell extract containing the AP-2 DNA-binding protein protected the AuPu2 DNA fragment from uracil DNA N-glycosylase activity and subsequent cleavage with heat and alkaline pH and thus, detects the presence of the AP-2 DNA-binding protein.

### Example 2

Use of RNase in Detecting an RNA-Binding Protein

RNase A can be used in conjunction with a macromolecular protection assay to detect an RNA-binding protein.

First, RNA transcripts are either chemically synthesized or they are prepared using a bacteriophage RNA polymerase transcription system. Further, they would be coupled to a label, for example, a fluorescent tag. The transcripts would then be incubated with a protein extract of interest to allow formation of one or more potential RNA-protein binding complexes. Subsequently, the sample would be incubated in the presence of RNase A, which cleaves free, single-stranded RNA. The digestion buffer for RNase reaction would include 300 mM NaCl, 10 mM Tris-HCl, pH 7.4, and 4 mM EDTA. The enzyme would be prepared at 10 mg/ml in 10 mM Tris-HCl, pH 7.4, and 1 mM EDTA containing 10 mM NaCl.

Detection of labeled RNA would be indicative of a protected RNA molecule and thus, an RNA-binding protein.

### Example 3

Use of RNase H in Analyzing Ribonucleoprotein Particles by Macromolecular Protection Assay In this example, the macromolecular protection assay would be used to analyze protein-binding domains in a ribonucleoprotein particle (RNP). More specifically, it could be used to detect specific protein domains having a binding interaction with an RNA of a RNP.

This example utilizes RNase H, an endonuclease that specifically hydrolyzes the phosphodiester bonds of RNA hybridized to DNA to produce 3′-OH and 5′-P terminated products. The enzyme does not degrade single-stranded nucleic acids, double-stranded DNA or double-stranded RNA. In addition, the RNA would be coupled to a detectable label, such as a fluorescent tag.

In this assay, the RNA component of a RNP and the corresponding deproteinized RNA would be targeted with an antisense DNA oligonucleotide that complements with a predetermined region of the RNA. If the oligonucleotide anneals to a complementary sequence of the RNA, RNase H will cleave the RNA within the double-stranded DNA/RNA region. The formation of the DNA/RNA hybrid will not occur however, in the presence of an RNP protein or protein domain that binds to the RNA. Thus, the RNA molecule would be protected from endonucleolytic cleavage by RNase H.

Detection of the label of intact RNA would indicate the formation of an RNA-protein binding complex and thus, the presence of an RNA-binding protein or domain thereof.

### Example 4

High-Throughput Detection of DNA-Binding Proteins Using the Macromolecular Protection Assay

This example demonstrates how the macromolecular protection assay can be carried out in a high-throughput manner to detect DNA-binding proteins. A plurality of protein samples and binding sequences could be screened simultaneously using this approach. The assay could achieve high-throughput, parallel processing of samples using microwell plates of varying densities and reaction volume capacities. Fluorescently-labeled DNA samples containing protein binding sequences of interest would be coupled onto the surface of microwell plate wells using biotin-streptavidin technology. More specifically, a streptavidin-coated microwell plate well could be used to bind biotin-labeled DNA.

Incubations at defined temperatures in a heat block, and subsequent wash/resuspension steps would be enabled by conventional microwell plate washers. Pipetting steps necessary for the addition of protein cell extracts and UNG would be performed manually.

Incorporation of fluorescent labels in the target DNA sequence allows for efficient detection and correspondingly, clear discrinination between un-protected and protected DNA sequences using commercially available microwell plate fluorescent detectors. (Molecular Devices). Detection of protected DNA would indicate a DNA-binding protein.

### Example 5

Automated Macromolecular Protection Assay for the Detection of DNA-Binding Proteins

This example demonstrates how the macromolecular protection assay can be carried out in a high-throughput, automated manner to detect DNA-binding proteins. A plurality of protein samples and binding sequences could be screened simultaneously using this approach. The assay could achieve high-throughput, parallel, and automated processing of samples using microwell plates of varying densities and reaction volume capacities. Fluorescently-labeled DNA samples containing protein binding sequences of interest would be coupled onto the surface of microwell plate wells using biotinstreptavidin technology. More specifically, a streptavidin-coated microwell plate well could be used to bind biotin-labeled DNA Alternatively, biotin-labeled DNA (with fluorescent tags) could be coupled to streptavidin-coated magnetic microparticles to facilitate downstream automated steps.

Incubations at defined temperatures in a heat block and subsequent wash/resuspension steps could be enabled by magnetic separation and wash solution dispensing/aspiration in an automated mode. All pipetting steps necessary for the addition of protein cell extracts and UNG would be performed automatically.

Incorporation of fluorescent labels in the target DNA sequence would allow for efficient detection and correspondingly, clear discrimination of un-protected vs. protected sequences. Thus, the detection of intact, protected DNA would indicate a DNA-binding protein.

### Example 6

Automated Macromolecular Protection Assay for the Detection of DNA-Binding Proteins with Reflex Testing

This example demonstrates how the macromolecular protection assay could be carried out in a high-throughput, automated manner to detect DNA-binding proteins. Further, it demonstrates how reflex testing can be performed in an automated mode to validate a DNA-protein complex.

A plurality of protein samples and binding sequences could be screened simultaneously using this approach. The assay could achieve high-throughput, parallel, and automated processing of samples using microwell plates of varying densities and reaction volume capacities. Fluorescently-labeled DNA samples containing protein binding sequences of interest would be coupled onto the surface of microwell plate wells using biotin-streptavidin technology. More specifically, a streptavidin-coated microwell plate well could be used to bind biotin-labeled DNA. Alternatively, biotin-labeled DNA (with fluorescent tags) could be coupled to streptavidin-coated magnetic microparticles to facilitate downstream automated steps.

Incubations at defined temperatures in a heat block and subsequent wash/resuspension steps could be enabled by magnetic separation and wash solution dispensing/aspiration in an automated mode. All pipetting steps necessary for the addition of protein cell extracts and UNG would be performed automatically.

Incorporation of fluorescent labels in the target DNA sequence would allow for efficient detection and correspondingly, clear discrimination of un-protected as compared to protected sequences. Thus, the detection of intact, protected DNA would indicate a potential DNA-binding protein.

The identified potential DNA-binding proteins could be further validated using reflex testing. Automation programming would be designed to retest the subset of extracts having potential DNA-binding proteins according to the following steps:

1. Automatically set up new binding reactions with DNA-binding sequence and extracts identified as having potential DNA-binding proteins such that the conditions for binding are more stringent (increased temperature or change buffer conditions).

2. Carry out the reactions in an automated mode.

3. Re-read the signals (e.g. fluorescence) produced by each binding reaction in an automated mode.

4. Identify reactions wherein a signal (e.g. fluorescence) remains.

5. Repeat steps 1 through 4 to increase confidence in detection of potential DNA-binding proteins.

Further validation could be afforded through the use of small-molecule inhibitors of DNA-binding proteins. Once a DNA-binding protein is identified, small-molecule inhibitors could be added to newly-prepared DNA-binding reactions of interest to see if there is specific inhibition of the binding complex by the small-molecule. The detection of specific inhibition further validates the characterization of a DNA-binding protein.

### Example 7

Automated Macromolecular Protection Assay for Screening Small-Molecule Inhibitors of DNA-Binding Proteins

This example demonstrates how the macromolecular protection assay can be carried out in a high-throughput, automated manner to detect inhibitors of DNA-binding proteins. Further, it demonstrates the reflex testing could be performed in an automated mode to validate a DNA-protein complex.

First, DNA-binding proteins could be identified according to Example 6. Subsequently, inhibitors of the DNA-binding proteins could be screened from a chemical library (comprising varying chemical species) or a plurality of chemical samples according to the following steps:

1. At least two different reactions would be set up.

a. In the first reaction, an extract having an identified DNA-binding protein or a purified DNA-binding protein of interest could be combined with its cognate DNA-binding sequence in a well of a microwell array. The DNA-binding sequence could have a biotin tag to facilitate downstream automation with streptavidin-coated magnetic microparticles. Further, the DNA would be labeled, for example, with a fluorescent tag.

b. In the second reaction, an extract and DNA-binding sequence according to step (a) could be combined with chemical species from a chemical library or a chemical sample having potential DNA-binding protein inhibitors.

2. The binding reactions would be allowed to proceed.

3. The signal, such as fluorescence, would be measured and compared from each reaction carried out according to step (1). A detected signal would indicate that the DNA-binding protein was able to bind to the DNA-binding sequence.

4. A lowering of the signal from the reaction containing the chemical sample would indicate a potential inhibitor.

Incubations at defined temperatures in a heat block and subsequent wash/resuspension steps could be enabled by magnetic separation and wash solution dispensing/aspiration in an automated mode. All pipetting steps necessary for the addition of protein cell extracts, UNG, and chemical samples would be performed automatically.

### Example 8

Conjugation of amino-polyA to IgGs Immobilized on Microwell Plates

The following example demonstrates that a polyA nucleotide, i.e. a repetitious sequence of adenine nucleotide residues, can be covalently linked vis-à-vis gluteraldehyde cross-linking to a protein (IgG) which is attached to a microwell plate. First, microwell plates are coated with high concentrations of IgGs by incubating the microwell plates with 100 microliters of a solution of 10 microgram mouse monoclonal IgG/100 microliter carbonate buffer (pH 9.6) for 6 hours at 37° C. The microwell plates were washed three times with phosphate-buffered saline (“PBS”) and reacted with 2% gluteraldehyde solution (in water) for 1 hour at 37° C. The microwell plates are again washed with PBS and then incubated for 2-6 hours with 250 nanograms of amine-polyA in 100 microliters of PBS along with 100 microgram/milliliter amino linker-reducing agent [PIERCE] . The microwell plates are then washed in PBS and allowed to dry for further evaluation.

The polyA-conjugated IgG was tested by hybridization. First, 1-100 nanograms of a biotin-labeled poly-T DNA was added to each well in the presence of 0.5 molar sodium thiocyanate (pH 9) at 37° C. and incubated for 1 hour. The wells were then washed three times with PBS-tween. Next, 100 microliters of streptavidin conjugated to horse radish peroxidase (“HRP”) was added to each well and incubated. Each well was then washed four times with PBS-tween followed by the addition of 3, 3′, 5, 5′-tetramethylbenzidine (“TMB”; BIOLOGICAL MIMETICS, INC., FREDERICK, Md.), i.e. the substrate for HRP which develops a deep blue soluble product when reacted with HRP labeled conjugates in microwell plates. The TMB was allowed to incubate for 15 minutes at room temperature, followed by the addition of 100 microliters of 1 normal solution of hydrochloric acid to halt further HRP activity. The optical density was determined at 450 nM wavelength for each microwell sample to determine the relative quantity of polyA present in each microwell. As a negative control, a microwell plate prepared only with bound IgG was tested.

Titration of the biotin-labeled poly-T DNA was carried out by adding different amounts poly-T DNA to separate wells of the microwell plates. The titration data showed that the poly-T DNA was saturating at a point between 10 and 100 ng as the optical density measurement was the same for both the 10 and 100 nanogram quantity of poly-T DNA. The negative control had significantly lower optical density measurements as expected as no poly-A DNA was introduced. The data is shown in Table 1.

### Example 9

Preparation of Anti-FITC (“fluorescein-5-isothiocyanate”) IgG Coated Microwell Plates

The following example demonstrates the coating of a microwell plate with anti-FITC as a generic capture agent for DNA labeled with FITC, a conventional fluorescent reporter molecule. IgGs were coated on plates in a passive manner. IgGs are known to bind to polystyrene although the precise mechanism is unknown. However, it is assumed that the binding is through hydrophobic domains of IgG, exposure of which can be enhanced in the presence of a high pH buffer of between 8.5-9.6.

A carbonate buffer was used in the preparation of the IgG coated microwell plates. The carbonate buffer was prepared by mixing 50-100 millimolar solutions of sodium bicarbonate [NaHCO₃] and sodium carbonate [Na₂CO₃] while checking to reach desired pH of 9.6.

The anti-FITC antibodies were obtained commercially (DAKO, AUSTRALIA). Both Goat-anti-FITC IgGs and Rabbit anti-FITC F(ab) were available. The antibodies were provided at a concentration of 2mg/ml and were diluted to the required coating concentration at 2 hours prior to use. The typical coating concentration of IgGs ranges between 5-20 micrograms/ml. 10 ug/ml was optimal for anti-FITC goat IgGs. Data is shown below in Table 1.

TABLE 1
Titration of Poly(T)

| Poly(T) | 0.1 ng | 1 ng | 10 ng | 100 ng |
|---|---|---|---|---|
| Poly A | 0.567 | 1.84 | 3 | 3 |
| IgG alone | 0.040 | 0.46 | 0.12 | 0.15 |

### Example 10

Macromolecular Protection Assay Using Anti-FITC Microwell Plates

In the following Example, the utility of the macromolecular protection assay, i.e. the method of the present invention to identify unknown DNA-binding proteins, is demonstrated using anti-FITC-coated microwell plates. The data show that the DNA binding protein, APII, as present in extracts of HeLa cells, is capable of protecting a DNA fragment containing APII binding site from cleavage by uracil N glycosylase (“UNG”). The detection method used was colorimetric and based on horse radish peroxidase (“HRP”) (see Tables 2 and 3, comparing rows 4-no extract-and 6-with extract).

First, dUTP-containing oligonucleotides were 5′ labeled with biotin. Complementary strands corresponding to promoter regions were synthesized with 5′ FITC. Double stranded DNA was prepared by heating 1 ug/ml DNA at 100° C. for 10 minutes followed by slow cooling at room temperature for 30 minutes.

DNA in UNG buffer was assayed independently with the following six reaction conditions:

(a) 10 ul of UNG Buffer;

(b) 2 units of UNG in 10 ul UNG buffer;

(c) 20 units of Endo III in Endo-Buffer;

(d) 2 units of UNG and 20 units of Endo II;

(e) 2.5 ul of Hela cell extract;

(f) 2 units of UNG, 20 units of Endo III and 2.5 ul of HeLa extract;

wherein the components were added in the following order:

1. DNA

2. HeLa cell extract, incubate for 15 minutes at 37° C.

3. UNG

4. Endo III

Twenty-five microliter aliquots of the mixture were tested at 30 minutes and 6 hours. 96-well microwell plates [COSTAR, CAMBRIDGE, Mass.] were coated with anti-FITC antibodies and blocked with an excess of non-specific proteins. The plates were dried at room temperature and stored at room temperature in sealed pouches containing desiccant.

Twenty-five microliters of the test sample was added to each well followed by 75 microliters of a blocking buffer [STABLEZYME]. The plates were incubated for 30 minutes at 37° C. and washed 4 times with PBS-Tween. 100 ul of a 1: 5000 dilution of Neutravidin-HRP [PIERCE, ROCKFORD, Ill.] was added per well and incubated for 30 minutes at 37° C. The plates were washed 4 times with PBS-Tween and 100 microliters of TMB substrate was added and incubated for 15 minutes at room temperature. The reaction was stopped by the addition of 100 microliters of 1N HCl and optical absorbance was read at 450 nM wavelength. The data is shown in Tables 2 and 3 below.

TABLE 2
Macromolecular Protection Assay - Thirty Minute Assay

| | Sample | Sample | Sample | |
|---|---|---|---|---|
| 30 Minutes Assay | 1 | 2 | 3 | Average |
| 1. Control | 1.862 | 2.156 | 2.048 | 2.022 |
| 2. UNG | 1.654 | 2.068 | 1.942 | 1.888 |
| 3. Endolll | 2.158 | 1.961 | 2.265 | 2.128 |
| 4. UNG + Endolll | 0.386 | 0.261 | 0.364 | 0.337 |
| 5. Hela Ext | 2.12 | 2.264 | 1.983 | 2.122 |
| 6. Hela + Ung + Endo | 2.041 | 2.224 | 1.969 | 2.078 |

TABLE 3
Macromolecular Protection Assay - Six Hour Assay

| | Sample | Sample | Sample | |
|---|---|---|---|---|
| 6 Hour Assay | 1 | 2 | 3 | Average |
| 1. Control | 1.981 | 2.196 | 1.869 | 2.015 |
| 2. UNG | 1.748 | 2.182 | 2.2 | 2.043 |
| 3. Endolll | 2.328 | 2.016 | 2.161 | 2.168 |
| 4. UNG + Endolll | 0.243 | 0.197 | 0.229 | 0.223 |
| 5. Hela Ext | 1.861 | 2.015 | 1.918 | 1.931 |
| 6. Hela + Ung + Endo | 1.883 | 2.182 | 1.951 | 2.005 |

Having thus described in detail preferred embodiments of the present invention, it is to be understood that the invention defined by the appended claims is not to be limited by particular details set forth in the above description as many apparent variations thereof are possible without departing from the spirit or scope thereof.

REFERENCES

1. Heyduk et al., “Molecular Beacons for Detecting DNA Binding Proteins,” Nature Biotechnology 20: 171-176 (2002).

2. Fried et al., “Equilibria and Kinetics of lac Repressor-Operator Interactions by Polyacrylamide Gel Electrophoresis,” Nucleic Acids Research 9:6505-6525 (1981).

3. Galas et al., “DNase Footprinting: A Simple Method for the Detection of Protein-DNA Binding Specificity,” Nucleic Acids Research 5:3157-3170 (1978).

4. Hill et al., “Fluorescence Approaches to Study of Protein-Nucleic Acid Complexation,” Methods in Enzymology 278: 390-416 (1997).

5. Speck et al., “From footprint to toeprint: a close-up of the DnaA box, the binding site for the bacterial initiator protein DnaA,” Nucleic Acids Research 25: 3242-3247 (1997)Devchand et al., “Uracil-DNA glycosylase as a probe for protein-DNA interactions,” Nucleic Acids Research 21: 3437-43 (1993).

6. Devchand et al., “Analysis of the tet repressor-operator interactions using the uracil-DNA glycosylase footprinting system,” Ann N Y Acad Sci 726: 309-11 (1994).

7. Pu et al., “Uracil interference, a rapid and general method for defining protein-DNA interactions involving the 5-methyl group of thymines: the GCN4-DNA complex,” Nucleic Acids Research 20: 771-775 (1992).

8. Xavier et al., “RNA as a drug target: methods for biophysical characterization and screening,” Trends in Biotechnology 18: 349-56 (2000).

9. DiDomenico et al., “COBAS AMPLICORTM: Fully Automated RNA and DNA Amplification and Detection System for Routine PCR,” Clinical Chemistry 42: 1915-1923 (1996).

10. Technical Bulletin No. 137, “Core Footprinting System,” Promega Corporation, Madison, Wis. p.1-15 (1999).

11. R. Rapley editor, “The Southwestern Assay,” The Nucleic Acid Protocols Handbook p.773-782 (2000).

12. R. Rapley editor, “The Gel Shift Assay for the Analysis of DNA-Protein Interactions,” The Nucleic Acid Protocols Handbook p.745-756 (2000).

13. R. Rapley editor, “DNase I Footprinting,” The Nucleic Acid Protocols Handbook p.729-735 (2000).

What is claimed is:

1. A method for detecting a nucleic acid-binding protein comprising the steps of:
(a) contacting a nucleic acid molecule comprising a nucleic acid-binding sequence with a protein sample potentially containing an unknown nucleic acid-binding protein under conditions sufficient to form a binding complex, wherein the binding complex protects bound nucleic acid from degradation;
(b) subjecting the binding complex to nucleic acid degradation conditions which degrade any unbound nucleic acid molecules; and
(c) detecting any bound nucleic acid, wherein detecting said bound nucleic acid indicates a nucleic acid-binding protein.

2. The method according to claim 1, further comprising the step of characterizing the nucleic acid-binding protein.

3. The method according to claim 2, wherein step of characterizing the nucleic acid-binding protein comprises a technique selected from the group consisting of immunodetection, mass spectrometry, amino acid sequencing, and enzymatic digestion of the DNA-binding protein.

4. The method according to claim 3, wherein the immunodetection comprises the steps of:
(a) contacting antibodies raised against known nucleic acid-binding proteins with the nucleic acid-binding protein under conditions sufficient to form a proteinantibody complex;
(b) detecting the protein-antibody complex; and
(c) characterizing the nucleic acid-binding protein of the protein-antibody complex.

5. The method according to claim 4, wherein the protein-antibody complex further comprises the bound nucleic acid molecule.

6. The method according to claim 1, wherein the protein sample of step (a) is selected from the group consisting of a cell extract, a purified protein, and a partially purified protein.

7. The method according to claim 6, wherein the cell extract is prepared from prokaryotic cells.

8. The method according to claim 6, wherein the cell extract is prepared from eukaryotic cells.

9. The method according to claim 8, wherein the eukaryotic cell is a mammalian cell.

10. The method according to claim 9, wherein the mammalian cell is a human cell.

11. The method according to claim 1, wherein the nucleic acid molecule is DNA.

12. The method according to claim 1, wherein the nucleic acid molecule is RNA.

13. The method according to claim 1, wherein the nucleic acid molecule of step (a) is selected from the group consisting of a mRNA, a synthetic RNA, cDNA, a PCR product, a DNA restriction fragment, a recombinant molecule, a genomic or partial genomic library, a cDNA library, a cDNA library, a synthetic DNA and combinations thereof.

14. The method according to claim 1, wherein the nucleic acid molecule contains a label.

15. The method according to claim 1, wherein step (c) comprises the step of detecting the label of the bound nucleic acid.

16. The method according to claim 5, wherein step of detecting the label comprises the step of using a detection system selected from the group consisting of a fluorescence detection system, a radioactive detection system, an enzyme-linked detection system, and a biotinylation detection system.

17. The method according to claim 4, wherein the label is a radioisotope.

18. The method according to claim 4, wherein the label is biotin.

19. The method according to claim 4, wherein the label is a fluorophore.

20. The method according to claim 7, wherein the radioisotope is selected from the group consisting of 32P, 33P, or 35S.

21. The method according to claim 9, wherein the fluorophore is selected from the group consisting of fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY.

22. The method of claim 8, wherein the step of detecting the label further comprises contacting the biotin with a binding partner.

23. The method of claim 2, wherein the binding partner is selected from the group of avidin, streptavidin, and anti-biotin antibody.

24. The method of claim 2, wherein the binding partner is conjugated to a fluorophore.

25. The method according to claim 4, wherein the fluorophore is selected from the group consisting of fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY.

26. The method according to claim 1, wherein step (c) comprises the steps of:
(cᵢ) contacting the bound nucleic acid with a nucleic acid dye;
(cᵢᵢ) detecting the nucleic acid dye.

27. The method according to claim 26, wherein the nucleic acid dye is selected from the group consisting of cyanine, cyanine-derivatives, PicoGreen, OliGreen, RiboGreen, TOTO dyes, intercalating dyes, ethidium bromide, propridium iodide, hexidium idodide, acridine orange, minor-groove-binding dyes, Hoeschst, and DAPI.

28. The method according to claim 1, wherein the nucleic acid degradation conditions are enzymatic.

29. The method according to claim 1, wherein the nucleic acid degradation conditions are enzymatic and physical.

30. The method according to claim 29, wherein the physical conditions comprise heat and alkali.

31. The method according to claim 1, wherein the nucleic acid molecule of step (a) further comprises a chemical modification enabling degradation of the nucleic acid molecule by an enzyme.

32. The method according to claim 31, wherein the chemical modification is introduced in the nucleotide base of one or more guanine, cytosine, thymine, or adenosine.

33. The method according to claim 32, wherein the enzyme that degrades the unbound nucleic acid molecule is a DNA N-glycosylase.

34. The method according to claim 32, wherein the enzyme that degrades the unbound nucleic acid molecule is selected from the group consisting of a DNA N-glycosylase, AP lyase, and combinations thereof.

35. The method according to claim 33, wherein the DNA N-glycosylase is selected from the group consisting of uracil DNA glycosylase, 3-methyladenine DNA glycosylase I, 3-methyladenine DNA glycosylase II, primidine hydrate DNA glycosylase (endonuclease III), formamidopyrimidine (FaPy) DNA glycosylase, thymine mismatch DNA glycosylase, and 8-oxoguanosine DNA glycosylase.

36. The method according to claim 34, wherein the AP lyase is selected from the group consisting of, pyrimidine hydrate DNA glycosylase (endonuclease III), formamidopyrimidine (FaPy) DNA glycosylase, exonuclease III and endonuclease IV.

37. The method according to claim 33, wherein degradation of the nucleic acid molecule comprises the steps of:
(bᵢ) contacting the nucleic acid molecule with the DNA N-glycosylase;
(bᵢᵢ) excising one or more nucleotide bases of the DNA molecule having the chemical modification;
(bᵢᵢᵢ) forming an AP site at each excised nucleotide base; and
(bᵢᵥ) exposing the nucleic acid molecule to heat and alkali conditions to cause break in the nucleic acid molecule at each AP site.

38. The method according to claim 34, wherein degradation of the nucleic acid molecule comprises the steps of:
(bᵢ) contacting the nucleic acid molecule with the DNA N-glycosylase;
(bᵢᵢ) excising one or more nucleotide bases of the nucleic acid molecule having the chemical modification;
(bᵢᵢᵢ) forming an AP site at each excised nucleotide base;
(bᵢᵥ) contacting the nucleic acid molecule with the AP lyase;
(bᵥ) forming a single-base lesion at each AP site; and
(bᵥᵢ) exposing the nucleic acid molecule to heat and alkali conditions to cause a break in the nucleic acid molecule at each single-base lesion.

39. The method according to claim 1, wherein step (b) comprises the step of contacting the binding complex with an enzyme in an amount sufficient to degrade unbound nucleic acid molecules.

40. The method according to claim 39, wherein the enzyme is a DNase.

41. The method according to claim 39, wherein the enzyme is an RNase.

42. The method according to claim 1, wherein the binding complex comprises a sequence-specific interaction between the nucleic acid-binding protein and the nucleic acid-binding sequence.

43. The method according to claim 1, wherein the binding complex comprises a non-sequence-specific interaction between the nucleic acid-binding protein and the nucleic acid-binding sequence.

44. A method for detecting a DNA-binding protein comprising the steps of:
(a) combining at least one DNA molecule, comprising a DNA-binding sequence and a label, with at least one protein sample potentially containing at least one unknown DNA-binding protein, in a reaction vessel comprising a plurality of wells;
(b) exposing the DNA molecule and the protein sample in the reaction vessel to conditions sufficient to form a binding complex, comprising the DNA-binding protein and a bound DNA molecule, wherein the binding complex protects the bound DNA molecule or fragment thereof from degradation;
(c) subjecting the binding complex to DNA degradation conditions, wherein any unbound DNA molecules are degraded; and
(d) detecting any bound DNA, wherein detecting said bound DNA indicates a DNA-binding protein, wherein said method is high-throughput.

45. The method according to claim 44, further comprising the step of characterizing the DNA-binding protein.

46. The method according to claim 45, wherein the step of characterizing the DNA-binding protein comprises a technique selected from the group consisting of immunodetection, mass spectrometry, amino acid sequencing, and enzymatic digestion of the DNA-binding protein.

47. The method according to claim 46, wherein the immunodetection comprises the steps of:
(a) contacting antibodies raised against known DNA-binding proteins with the DNA-binding protein under conditions sufficient to form a protein-antibody complex;
(b) detecting the protein-antibody complex; and
(c) characterizing the DNA-binding protein of the protein-antibody complex.

48. The method according to claim 47, wherein the protein-antibody complex further comprises the bound DNA molecule.

49. The method according to claim 44, wherein the method is automated.

50. The method according to claim 44, wherein step (a) further comprises binding said at least one DNA molecule to a magnetic microparticle in said reaction vessel.

51. The method according to claim 44, wherein the protein sample of step (a) is selected from the group consisting of a cell extract, a purified protein, and a partially purified protein.

52. The method according to claim 51, wherein the cell extract is prepared from prokaryotic cells.

53. The method according to claim 52, wherein the cell extract is prepared from eukaryotic cells.

54. The method according to claim 53, wherein the eukaryotic cell is a mammalian cell.

55. The method according to claim 54, wherein the mammalian cell is a human cell.

56. The method according to claim 44, wherein the DNA molecule of step (a) is selected from the group consisting of a cDNA, a PCR product, a DNA restriction fragment, a recombinant molecule, a genomic or partial genomic library, a cDNA library, a synthetic DNA and combinations thereof.

57. The method according to claim 44, wherein the DNA molecule contains a label.

58. The method according to claim 44, wherein step (d) comprises the step of detecting the label the bound DNA.

59. The method according to claim 58, wherein step of detecting the label comprises the step of using a detection system selected from the group consisting of a fluorescence detection system, a radioactive detection system, an enzyme-linked detection system, and a biotinylation detection system.

60. The method according to claim 58, wherein the label is a radioisotope.

61. The method according to claim 58, wherein the label is biotin.

62. The method according to claim 58, wherein the label is a fluorophore.

63. The method according to claim 60, wherein the radioisotope is selected from the group consisting of 32P, 33P, or 35S.

64. The method according to claim 62, wherein the fluorophore is selected from the group consisting of fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY.

65. The method of claim 61, wherein the step of detecting the label further comprises contacting the biotin with a binding partner.

66. The method according to claim 65, wherein the binding partner is selected from the group of avidin, streptavidin, and anti-biotin antibody.

67. The method according to claim 65, wherein the binding partner is conjugated to a fluorophore.

68. The method according to claim 67, wherein the fluorophore is selected from the group consisting of fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY.

69. The method according to claim 44, wherein step (e) comprises the steps of:
(dᵢ) contacting the bound DNA with a nucleic acid dye;
(dᵢᵢ) detecting the nucleic acid dye.

70. The method according to claim 69, wherein the nucleic acid dye is selected from the group consisting of cyanine, cyanine-derivatives, PicoGreen, OliGreen, RiboGreen, TOTO dyes, intercalating dyes, ethidium bromide propridium iodide, hexidium idodide, acridine orange, minor-groove-binding dyes, Hoeschst, and DAPI.

71. The method according to claim 44, wherein the DNA degradation conditions are enzymatic.

72. The method according to claim 44, wherein the DNA degradation conditions are enzymatic and physical.

73. The method according to claim 72, wherein the physical conditions comprise heat and alkali.

74. The method according to claim 44, wherein the DNA molecule of step (a) further comprises a chemical modification enabling degradation of the DNA molecule by an enzyme.

75. The method according to claim 74, wherein the chemical modification is introduced in the nucleotide base of one or more guanine, cytosine, thymine, or adenosine.

76. The method according to claim 74, wherein the enzyme that degrades the unbound DNA molecule is a DNA N-glycosylase.

77. The method according to claim 74, wherein the enzyme that degrades the unbound DNA molecule is selected from the group consisting of a DNA N-glycosylase and an AP lyase.

78. The method according to claim 76, wherein the DNA N-glycosylase is selected from the group consisting of uracil DNA glycosylase, 3-methyladenine DNA glycosylase I, 3-methyladenine DNA glycosylase II, pyrimidine hydrate DNA glycosylase (endonuclease III), formamidopyrimidine (FaPy) DNA glycosylase, thymine mismatch DNA glycosylase, and 8-oxoguanosine DNA glycosylase.

79. The method according to claim 77, wherein the AP lyase is selected from the group consisting of, pyrimidine hydrate DNA glycosylase (endonuclease III), formamidopyrimidine (FaPy) DNA glycosylase, exonuclease III and endonuclease IV.

80. The method according to claim 76, wherein degradation of the DNA molecule comprises the steps of:
(cᵢ) contacting the DNA molecule with the DNA N-glycosylase;
(cᵢᵢ) excising one or more nucleotide bases of the DNA molecule having the chemical modification;
(cᵢᵢᵢ) forming an AP site at each excised nucleotide base; and
(cᵢᵥ) exposing the DNA molecule to heat and alkali conditions to cause a break in the DNA molecule at each AP site.

81. The method according to claim 161, wherein degradation of the DNA molecule comprises the steps of:
(cᵢ) contacting the DNA molecule with the DNA N-glycosylase;
(cᵢᵢ) excising one or more nucleotide bases of the DNA molecule having the chemical modification;
(cᵢᵢᵢ) forming an AP site at each excised nucleotide base;
(cᵢᵥ) contacting the DNA molecule with the AP lyase;
(cᵥ) forming a single-base lesion at each AP site; and
(cᵥᵢ) exposing the DNA molecule to heat and alkali conditions to cause a break in the DNA molecule at each single-base lesion.

82. The method according to claim 44, wherein step (c) comprises the step of contacting the binding complex with an enzyme in an amount sufficient to degrade unbound DNA molecules.

83. The method according to claim 82, wherein the enzyme is a DNase.

84. The method according to claim 44, wherein the binding complex comprises a sequence-specific interaction between the DNA-binding protein and the DNA-binding sequence.

85. The method according to claim 44, wherein the binding complex comprises a non-sequence-specific interaction between the DNA-binding protein and the DNA-binding sequence.

86. A method for detecting an inhibitor of a nucleic acid-binding protein comprising the steps of:
(a) preparing a first reaction mixture by combining at least one protein sample potentially containing one or more unknown nucleic acid-binding proteins with a nucleic acid molecule, comprising a label and nucleic acid-binding sequence, in a first well of a reaction vessel under conditions sufficient to form a binding complex, comprising the nucleic acid-binding protein and the nucleic acid-binding sequence;
(b) preparing a second reaction mixture by combining the protein sample of the first reaction with a nucleic acid molecule of the first reaction and at least one chemical sample potentially containing one or more inhibitors of the nucleic acid-binding protein in a second well of a reaction vessel under conditions sufficient to form the binding complex, wherein the inhibitor prevents the formation of the binding complex;
(c) treating the first and second reaction mixtures in a manner sufficient to degrade unbound nucleic acid molecules; and
(d) detecting the amount of intact nucleic acid of both first and second reaction mixtures, wherein less amount of intact nucleic acid of said second reaction mixture indicates the presence of an inhibitor.

87. The method according to claim 86, further comprising the step of isolating the inhibitor.

88. The method according to claim 86, wherein the protein sample of step (a) is selected from the group consisting of a cell extract, a purified protein, and a partially purified protein.

89. The method according to claim 88, wherein the cell extract is prepared from prokaryotic cells.

90. The method according to claim 88, wherein the cell extract is prepared from eukaryotic cells.

91. The method according to claim 90, wherein the eukaryotic cell is a mammalian cell.

92. The method according to claim 91, wherein the mammalian cell is a human cell.

93. The method according to claim 86, wherein the nucleic acid molecule of step (a) is selected from the group consisting of a MRNA, cDNA, a PCR product, a DNA restriction fragment, a recombinant molecule, a genomic or partial genomic library, a cDNA library, a synthetic DNA, a synthetic RNA and combinations thereof.

94. The method according to claim 86, wherein the nucleic acid molecule contains a label.

95. The method according to claim 86, wherein step (d) comprises the step of detecting the label of the intact nucleic acid.

96. The method according to claim 95, wherein step of detecting the label comprises the step of using a detection system selected from the group consisting of a fluorescence detection system, a radioactive detection system, an enzyme-linked detection system, and a biotinylation detection system.

97. The method according to claim 95, wherein the label is a radioisotope.

98. The method according to claim 95, wherein the label is biotin.

99. The method according to claim 95, wherein the label is a fluorophore.

100. The method according to claim 97, wherein the radioisotope is selected from the group consisting of 32P, 33P, or 35S.

101. The method according to claim 99, wherein the fluorophore is selected from the group consisting of fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY.

102. The method according to claim 98, wherein the step of detecting the label further comprises contacting the biotin with a binding partner.

103. The method according to claim 102, wherein the binding partner is selected from the group of avidin, streptavidin, and anti-biotin antibody.

104. The method according to claim 103, wherein the binding partner is conjugated to a fluorophore.

105. The method according to claim 104, wherein the fluorophore is selected from the group consisting of fluorescein, fluorescein-derivative, rhodamine, rhodamine-derivative, Texas Red, Oregon Green, Alexa Fluor, Cascade Blue, Tetramethylrhodamine, Pacific Blue, SYBR, and BODIPY.

106. The method according to claim 86, wherein step (d) comprises the steps of:
(dᵢ) contacting the intact nucleic acid with a nucleic acid dye;
(dᵢᵢ) detecting the nucleic acid dye.

107. The method according to claim 106, wherein the nucleic acid dye is selected from the group consisting of cyanine, cyanine-derivatives, PicoGreen, OliGreen, RiboGreen, TOTO dyes, intercalating dyes, ethidium bromide, propridium iodide, hexidium idodide, acridine orange, minor-groove-binding dyes, Hoeschst, and DAPI.

108. The method according to claim 86, wherein the nucleic acid degradation conditions are enzymatic.

109. The method according to claim 86, wherein the nucleic acid degradation conditions are enzymatic and physical.

110. The method according to claim 109, wherein the physical conditions comprise heat and alkali.

111. The method according to claim 86, wherein the nucleic acid molecule of step (a) further comprises a chemical modification enabling degradation of the nucleic acid molecule by an enzyme.

112. The method according to claim 111, wherein the chemical modification is introduced in the nucleotide base of one or more guanine, cytosine, thymine, or adenosine.

113. The method according to claim 111, wherein the enzyme that degrades the unbound nucleic acid molecule is a DNA N-glycosylase.

114. The method according to claim 111, wherein the enzyme that degrades the unbound nucleic acid molecule is selected from the group consisting of an DNA N-glycosylase, an AP lyase, and combinations thereof.

115. The method according to claim 113, wherein the DNA N-glycosylase is selected from the group consisting of uracil DNA glycosylase, 3-methyladenine DNA glycosylase I, 3-methyladenine DNA glycosylase II, pyrimidine hydrate DNA glycosylase (endonuclease III), formamidopyrimidine (FaPy) DNA glycosylase, and thymine mismatch DNA glycosylase.

116. The method according to claim 114, wherein the AP lyase is selected from the group consisting of, pyrimidine hydrate DNA glycosylase (endonuclease III), formamidopyrimidine (FaPy) DNA glycosylase, exonuclease III and endonuclease IV.

117. The method according to claim 113, wherein degradation of the nucleic acid molecule comprises the steps of:
(dᵢ) contacting the nucleic acid molecule with the DNA N-glycosylase;
(dᵢᵢ) excising one or more nucleotide bases of the nucleic acid molecule having the chemical modification;
(dᵢᵢᵢ) forming an AP site at each excised nucleotide base; and
(dᵢᵥ) exposing the nucleic acid molecule to heat and alkali conditions to cause a break in the nucleic acid molecule at each AP site.

118. The method according to claim 114, wherein degradation of the nucleic acid molecule comprises the steps of:
(dᵢ) contacting the nucleic acid molecule with the DNA N-glycosylase;
(dᵢᵢ) excising one or more nucleotide bases of the nucleic acid molecule having the chemical modification;
(dᵢᵢᵢ) forming an AP site at each excised nucleotide base;
(dᵢᵥ) contacting the nucleic acid molecule with the AP lyase;
(dᵥ) forming a single-base lesion at each AP site; and
(dᵥᵢ) exposing the nucleic acid molecule to heat and alkali conditions to cause a break in the nucleic acid molecule at each single-base lesion.

119. The method according to claim 86, wherein step (e) comprises the step of contacting the first and second reaction mixtures with an enzyme in an amount sufficient to degrade unbound nucleic acid molecules.

120. The method according to claim 119, wherein the enzyme is a DNase

121. The method according to claim 119, wherein the enzyme is a RNase.

122. The method according to claim 86, wherein the binding complex comprises a sequence-specific interaction between the nucleic acid-binding protein and the nucleic acid-binding sequence.

123. The method according to claim 86, wherein the binding complex comprises a non-sequence-specific interaction between the nucleic acid-binding protein and the nucleic acid-binding sequence.

124. The method according to claim 86, wherein the nucleic acid molecule is DNA or RNA.

125. The methods according to claims 1, 44, and 86 are automated

126. The methods according to claims 1, 44 and 86 are high-throughput

127. A kit for carrying out the method according to claim 1, comprising protein samples potentially containing an unknown nucleic acid-binding protein, a nucleic acid molecule comprising a nucleic acid-binding sequence, components for preparing the nucleic acid molecule, a label, an enzyme to attach the label to the nucleic acid molecule, an enzyme to degrade or facilitate the degradation of unbound nucleic acid, and reagents to assist in the degradation of unbound nucleic acid, and a detection system to detect the label of the nucleic acid

128. The method according to claim 44, wherein the reaction vessel is a microtiter plate

129. The method according to claim 44, wherein the reaction vessel comprises an array of wells ranging from 1 to 5,000.

130. The method according to claim 44, wherein the reaction vessel comprises an array of wells ranging from 1 to 1,000.

131. The method according to claim 44, wherein the reaction vessel comprises an array of wells ranging from 1 to 500.

132. The method according to claim 127, wherein the microtiter plate comprises 96 wells.

133. The method according to claim 127, wherein the microtiter plate comprises 384 wells.

134. The method according to claim 44, wherein the reaction vessel is a strip well unit.

135. The method according to claim 44, wherein the reaction vessel is a planar well unit.

136. The method according to claim 44, wherein the reaction vessel is a chip well unit.

137. The method according to claims 44, wherein the method further comprises step (e), wherein step (e) comprises validating the detection of the nucleic-acid binding protein.

138. The method according to claim 138, wherein step (e) further comprises the steps of:
(eᵢ) potential DNA-binding proteins contained in the one or more protein samples are detected according to steps (a) through (d) wherein the DNA and the protein samples are combined according to step (a) and exposed to conditions sufficient to form a binding complex according to step (b), wherein said conditions are low-stringency conditions sufficient to form non-specific binding complexes;
(eᵢᵢ) the protein samples containing a detected DNA-binding protein are contacted again with the DNA molecule and exposed to conditions that are of higher-stringency than step (eᵢ) that are sufficient to form a more highly-specific binding complex comprising the DNA-binding protein bound to its preferred DNA-binding sequence, wherein the binding complex protects the bound DNA molecule or fragment thereof from degradation;
(eᵢᵢᵢ) subjecting the binding complexes to DNA degradation conditions, wherein any unbound DNA molecules are degraded;
(eᵢᵥ) detecting any bound DNA, wherein detecting said bound DNA indicates the presence of a DNA-binding protein;
(eᵢᵢ) repeating steps (eᵢᵢ) to (eᵢᵥ), wherein the stringency of the conditions of step (eᵢᵢ) are progressively increased each round.

139. A method for detecting a nucleic acid-binding protein comprising the steps of:
(a) contacting a nucleic acid molecule comprising a nucleic acid-binding sequence with a protein sample potentially containing an unknown nucleic acid-binding protein under conditions sufficient to form a binding complex, wherein the binding complex protects bound nucleic acid from degradation;
(b) subjecting the binding complex to nucleic acid degradation conditions which degrade any unbound nucleic acid molecules; and
(c) detecting any bound nucleic acid, wherein detecting said bound nucleic acid indicates a nucleic acid-binding protein;
wherein steps (a) through (c) are carried out in a well of a microtiter plate.

140. The method of claim 139, wherein the nucleic acid molecule is hybridized to a generic capture reagent.

141. The method of claim 140, wherein the generic capture reagent is poly(A)-IgG, wherein poly(A) is hybridized to the nucleic acid molecule and IgG is coupled to the well of the microtiter plate.

142. The method of claim 141, wherein the nucleic acid molecule of claim 140 is covalently coupled to FITC and the generic capture reagent of claim 141 is anti-FITC, wherein the anti-FITC is coupled to the well of the microtiter plate.

143. The method of claim 139, wherein the method is high-throughput.

144. The method of claim 139, wherein the method is automated.

145. A method of transmitting a result of an assay, said assay comprising the steps of:
(a) contacting a nucleic acid molecule comprising a nucleic acid-binding sequence with a protein sample potentially containing an unknown nucleic acid-binding protein under conditions sufficient to form a binding complex, wherein the binding complex protects bound nucleic acid from degradation;
(b) subjecting the binding complex to nucleic acid degradation conditions which degrade any unbound nucleic acid molecules; and
(c) detecting any bound nucleic acid, wherein detecting said bound nucleic acid indicates a nucleic acid-binding protein;
(d) characterizing the nucleic acid-binding protein wherein the characterized nucleic acid-binding protein constitutes the result of said assay.

146. The method of claim 145 wherein steps (a) through (c) are carried out in a microtiter plate.

147. The method according to claim 145, wherein step (d) further comprises a technique selected from the group consisting of immunodetection, mass spectrometry, amino acid sequencing, and enzymatic digestion of the DNA-binding protein.

148. A reaction vessel for carrying out the method of claim 1 wherein the reaction vessel is coated with IgG antibodies coupled to amino-poly(A) and the nucleic acid molecule of claim 1 comprises a T-tail.

149. A reaction vessel for carrying out the method of claim 1 wherein the reaction vessel is coated with anti-FITC antibodies and the nucleic acid molecule of claim 1 comprises FITC.

150. The reaction vessel of claims 148 and 149, wherein the reaction vessel is a microwell plate.

151. The kit for carrying out the method according to claim 1 comprising a DNA N-glycosylase, a DNA N-glycosylase reaction buffer, reagents to assist in the degradation of unbound nucleic acid, the reaction vessel of claim 148, and optional instructions.

152. The kit for carrying out the method according to claim 1 comprising a DNA N-glycosylase, a DNA N-glycosylase reaction buffer, reagents to assist in the degradation of unbound nucleic acid, the reaction vessel of claim 149, and optional instructions.
